(19) United States
(12) Patent Application Publication
(10) Pub. No.: US 2026/0021314 A1
(43) Pub. Date: Jan. 22, 2026

(54) METHODS FOR USING MEDICAL DEVICE DATA FOR INSIGHTS IN MEDICAL DEVICE USE AND PERFORMANCE

(71) Applicant: Stryker Corporation, Portage, MI (US)

(72) Inventors: Tyson G. Taylor, Bothell, WA (US); Fred W. Chapman, Newcastle, WA (US); Robert G. Walker, Seattle, WA (US); Robert P. Marx, Kent, WA (US); Rose Tingwei Yin, Bellevue, WA (US)

(73) Assignee: Stryker Corporation, Portage, MI (US)

(21) Appl. No.: 19/275,029

(22) Filed: Jul. 21, 2025

Related U.S. Application Data
(60) Provisional application No. 63/674,034, filed on Jul. 22, 2024.

(51) Int. Cl. A61N 1/39; G06F 21/62; G16H 40/63

(52) U.S. Cl. CPC A61N 1/3937; G06F 21/6254; G16H 40/63

(57) ABSTRACT

An example method includes generating, at a medical device, first data comprising (i) an identifier of a subject being monitored or treated by the medical device, (ii) data indicating a state of the medical device during a rescue event, (iii) data indicating a user of the medical device during the rescue event, and storing the first data in a memory associated with the medical device. The example method further includes generating second data by de-identifying the first data and transmitting the second data to an external device configured to receive the de-identified data from a fleet of medical devices and identify a trend associated with the de-identified data using a computing model(s).

### CROSS-REFERENCE TO RELATED APPLICATION

This application claims priority to U.S. Provisional App. No. 63/674,034, which was filed on Jul. 22, 2024 and is hereby incorporated by reference herein in its entirety.

### BACKGROUND

Medical devices can be used to monitor patients and/or to administer treatments to patients. Medical devices used in emergency situations must enable rapid and correct use for timely treatment of patients experiencing life-threatening emergencies. However, it is not always clear if or how a medical device is being misused during a rescue event. Current sources of information that may provide insight into the use of various medical devices are limited. Gathering data to improve medical devices is mostly a manual process and generally performed only when a question of particular interest arises due to the processing being costly and time consuming.

### BRIEF DESCRIPTION OF THE DRAWINGS

FIG. 1 illustrates an example environment for de-identifying data generated by medical device(s) and transmitting the de-identified data to an external computing device.

FIG. 2 illustrates example signaling for facilitating communication between devices.

FIG. 3 illustrates an example of an external defibrillator configured to perform various functions described herein.

FIG. 4 illustrates a chest compression device configured to perform various functions described herein.

FIG. 5 illustrates an example process for de-identifying privileged data generated at a medical device and transmitting the de-identified data to an external device.

FIG. 6 illustrates an example process 600 for determining a trend indicating misuse of a fleet of medical devices by analyzing data received from the fleet of medical devices.

### DETAILED DESCRIPTION

Medical devices used in emergency situations benefit from rapid and correct use for timely treatment of patients experiencing life-threatening emergencies. However, previous sources of information regarding use of medical devices are limited to user interviews, observation of simulated events, and/or examination of medical device data associated with complaints. Clinicians who use medical devices, such as monitor-defibrillators, may be unaware that they are misusing the medical device or not using the device optimally. Additionally, it would be beneficial to track the performance of clinical algorithms, such as a shock advisory algorithm utilized by an automated external defibrillator (AED), to ensure continued high performance despite various types of patient conditions that were not present during initial development.

Various implementations described herein address these and other problems by automating the synthesis of data that provides insight about how medical devices are used, how the medical devices perform from data captured by the medical devices, and data about new or rare medical conditions. Moreover, it may be beneficial to synthesize this data without exposing personally identifying data associated with patients that are monitored and treated by the medical devices. For instance, a fleet of medical devices may be configured to deidentify (at the device level) privileged data to ensure that the patient data, user operational data, and/or device data no longer contains protected personally identifiable information. The deidentified data is transmitted to an external computing device(s) where a trend(s) can be identified using computing model(s). A trend may indicate misuse of a particular medical device, a type of medical device, and/or a fleet of medical devices. For example, analysis of the deidentified data may reveal trends and changes in physiological parameters that occur gradually or abruptly, trends that indicate disease progression that was not previously known, trends that indicate responses to treatment, recovery from illness, or worsening of symptoms. In some examples, analysis of environmental data associated with a fleet of medical devices provides insight into trend(s) that may indicate changes in physiological parameters due to geographic location, climate variations, seasonal variations in response to environmental factors such as temperature, humidity, sunlight exposure, and/or allergen levels.

Various specific examples of implementations of the present disclosure will be described. In some instances, analysis of the deidentified data may uncover a trend that indicate users (e.g., a threshold number of users) fail to recognize lead-wire reversal when it occurs and users may be unaware of necessary corrective action that is needed to optimize electrocardiogram (ECG) quality. As such, there may be a need to alert users to these types of errors (or change a currently implemented alert). Additionally, some medical devices may alert a user via a message that an artifact is detected, but the alert may not be optimal (e.g., it may not inform the clinician as to the particular cause of the artifact). As an additional example, analysis of the deidentified data may uncover a trend indicating misuse of a fleet of medical devices including a delay between treatment recommendations output by the fleet of medical devices and treatments administered to multiple subjects or non-indicated treatments administered to the multiple subjects. These and additional examples are discussed in further detail below.

Implementations of the present disclosure are directed to particular improvements in the technical field of medical device management and medical care. Previous systems do not allow for large scale collection of medical device use and operational data due to the privileged nature of some of the data received and/or generated. However, much of this information is highly beneficial for analysis and research as it is difficult to recreate rare events that may occur during an emergency within a lab setting. For example, subjects experiencing rare cardiac events (e.g., ventricular fibrillation (VF) storm, electromechanical dissociation (EMD), etc.) or have rare cardiac conditions (e.g., Brugada syndrome, Catecholaminergic Polymorphic Ventricular Tachycardia (CPVT), Arrhythmogenic Right Ventricular Dysplasia (ARVD), etc.) may affect the performance and/or effectiveness of shock advisory algorithms or other treatments administered by medical devices. It is difficult to collect reliable medical device performance data and/or medical device use data associated with these rare cardiac events and/or rare cardiac conditions due to the difficulty in replicating these events in a research setting. Additionally, misuse of a medical device is not always apparent, particularly if a medical device is misused by a group of users in a particular hospital or geographic region.

Physiological parameter data generated and/or received by a medical device is associated with personally identifiable data and is therefore privileged. Implementations of the present disclosure are directed to de-identifying data associated with the use and performance of a fleet of medical devices at the device level and prior to sending the data to a computing device for analysis. For example, a de-identification component associated with the medical device may be configured to encrypt the privileged data using an encryption algorithm and a unique encryption key. This enables for the continuous monitoring of a large amount of data generated in a short time span.

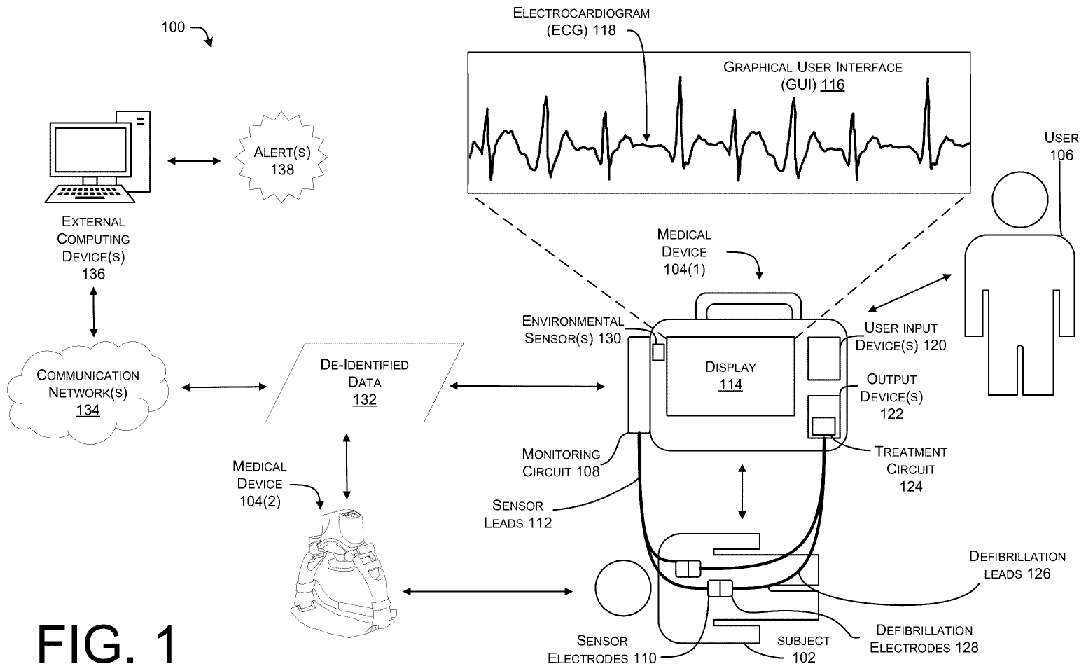

FIG. 1 illustrates an example environment 100 for de-identifying data generated by a medical device (at the device level) and transmitting the de-identified data to an external computing device(s) 136. As illustrated, the environment includes a rescue scene. In some examples, the rescue scene may be in a clinical environment (e.g., a hospital) or a non-clinical environment (e.g., the scene of an accident). In some cases, the subject 102 may be experiencing cardiac arrest, respiratory arrest, or some other dangerous medical condition. The subject 102 is being monitored and/or treated by a user 106 (e.g., an emergency medical technician (EMT) professional, care provider, etc.) utilizing one or more medical devices. The user 106 may be utilizing one or more medical devices to evaluate, treat and/or monitor a condition of the subject 102. For example, the user 106 may utilize a first medical device 104(1) (e.g., a monitor-defibrillator) and a second medical device 104(2) (e.g., a chest compression device). In some instances, additional users may be assisting user 106.

According to some implementations, the medical device 104(1) is a defibrillator. For example, the medical device 104(1) may be an external defibrillator, such as an automated external defibrillator (AED) or a monitor-defibrillator. In some examples, the medical device 104(1) may be a portable medical device. A user 106 may utilize the medical device 104(1) to evaluate, treat, and/or monitor a condition of the subject 102. In some examples, the user 106 may utilize additional medical device(s) (e.g., medical device 104(2)) to evaluate, treat, and/or monitor the subject 102. For example, the user 106 may utilize one or more of an additional monitor-defibrillator, a medical imaging device, an ultrasound monitor, a standalone ECG monitor, chest compression device, ventilator, pulse oximeter, capnograph, continuous glucose monitor (CGM), blood pressure monitor, portable X-ray machine, transport cot or bed, and/or any other medical device or equipment configured to assess, treat, and/or monitor an individual. Although illustrated in FIG. 1 as separate individuals, in some cases, the subject 102 is the user 106.

The medical device 104(1) (e.g., a monitor-defibrillator) may include a monitoring circuit 108, sensor(s) (e.g., parameter sensor(s), sensor electrodes 110, environmental sensor(s) 130, etc.), a display 114, a user input device 120, an output device 122, and/or a treatment circuit 124).

A monitoring circuit 108, for instance, detects one or more physiological parameters of the subject 102 via the sensor(s). As used herein, the term “physiological parameter,” and its equivalents, may refer to an indication of a subject's health including, for instance, an ECG, an impedance (e.g., transthoracic impedance), a force administered to the subject 102, a blood pressure, an airway parameter (e.g., a partial pressure of carbon dioxide, a partial pressure of oxygen, a capnograph, an end tidal gas parameter (e.g., end-tidal CO₂ (EtCO₂)), a flow rate, etc.), a blood oxygenation (e.g., a pulse oximetry value, a regional oximetry value (e.g., cerebral regional tissue oxygen saturation), etc.), an electroencephalogram (EEG), an electromyogram (EMG), a temperature, a heart sound, a blood flow rate, a physiological geometry (e.g., a shape of a blood vessel, an inner ear shape, etc.), a heart rate, a pulse rate, a chest compression position, or the like.

The monitoring circuit 108 includes and/or is communicatively coupled to a sensor(s). The sensor(s) may be configured to detect at least one physiological parameter of the subject 102. For example, a sensor may include at least one of electrodes (e.g., sensor electrodes 110), a detection circuit, defibrillator pads, a force sensor, a blood pressure cuff, an ultrasound-based blood pressure sensor, an invasive (e.g., intra-arterial) blood pressure sensor (e.g., including a cannula inserted into the subject 102), a gas sensor (e.g., a carbon dioxide and/or oxygen sensor), a flowmeter, a pulse oximetry sensor, a regional oximetry sensor, etc. In some instances, the medical device 104(1) may include a thermometer and/or a microphone.

In some cases, the physiological parameters indicate a condition of a heart of the subject 102. According to various examples, the physiological parameters include a voltage generated by the heart of the subject 102, a pulse oximetry level of the subject 102, or any combination thereof. According to some examples, the physiological parameters detected by the monitoring circuit 108 include an electrical signal output by the heart of the subject 102. For example, sensor electrodes 110 receive an electrical signal (e.g., the voltage) output by the heart of the subject 102. The sensor electrodes 110 may be adhered to the subject 102 via an adhesive. The sensor electrodes 110 receive an electrical signal (e.g., the voltage) output by the heart of the subject 102. Sensor leads 112 electrically couple the sensor electrodes 110 to the monitoring circuit 108. In some cases, the monitoring circuit 108 is configured to convert the electrical signal into digital data indicative of an ECG 118 (also referred to as an “ECG signal 118”) of the subject 102. Although FIG. 1 illustrates a pair of sensor electrodes 110, in some examples, the monitoring circuit 108 receives electrical signals indicative of the ECG 118 from three or more sensor electrodes 110. For example, a 12-lead ECG is obtained from ten sensor electrodes 110 placed on the skin of the subject 102. In some examples, the monitoring circuit 108 includes an analog to digital converter.

In some examples, the monitoring circuit 108 includes one or more ports configured to receive analog signals indicative of the physiological parameters. The one or more ports can be electrically coupled to one or more sensors (e.g., parameter sensor).

The medical device 104(1) may include a display 114 configured to graphically output physiological parameters, alerts, messages, instructions, etc. As shown in the example of FIG. 1, the display 114 outputs a graphical user interface (GUI) 116 that includes at least a portion of the ECG 118 of the subject 102. The ECG 118 includes a first axis corresponding to time and a second axis corresponding to voltage. In some cases, the GUI 116 includes multiple plots corresponding to different (e.g., 12) leads of the ECG 118. In some instances, the display 114 may present an alert, instruction, recommendation, etc. to the user 106. In some examples, the alert may be a visual and/or aural alert. In some instances, the GUI 116 may notify users of a detected artifact, types of artifacts, lead(s) affected, and electrodes causing a problem, lead wire reversal, if present, or any other type of problem, warning, recommendation, instruction, etc. The display may present physiological parameters in a manner that is easily digested and understood while also conveying a reliability of such data, without filling or cluttering a display with background or additional data that is typically used in a manual method to evaluate the data on the display.

The medical device 104(1) includes a user input device(s) 120 and an output device(s) 122. The user input device(s) 120 and the output device(s) 122 function as an interface between a user and the medical device 104(1). The input device(s) 120 is configured to receive an input from a user and includes at least one of a keypad, a cursor control, a touch-sensitive display, a voice input device (e.g., a microphone), a haptic feedback device (e.g., a gyroscope), or any combination thereof. The output device(s) 122 includes at least one of a display, a speaker, a haptic output device, a printer, or any combination thereof. In some implementations, the input device(s) 120 includes one or more touch sensors, and the output device(s) 122 includes a display screen, and the touch sensor(s) are integrated with the display screen.

In at least one example, the medical device 104(1) is a monitor-defibrillator configured to output a defibrillation shock and/or a pacing signal to the subject 102. The output device 122 is connected to a set of defibrillation leads 126. The defibrillation leads 126 are connected to defibrillation electrodes 128 that are in contact with the skin of the subject 102. According to some examples, the defibrillation electrodes 128 are integrated with, or at least partially in contact with, at least some of the sensor electrodes 110. In some cases, the output device 122 includes a treatment circuit 124 configured to selectively output a voltage (e.g., a defibrillation shock) across the defibrillation electrodes 128. For example, the output device 122 (s) may include a capacitor configured to store a voltage and a discharge circuit configured to discharge the voltage across the defibrillation electrodes 128. The voltage is applied over the heart of the subject 102 and depolarizes cells within the heart. The voltage may cause the heart to, at least eventually, restore a healthy heart rhythm.

In some examples, a second medical device 104(2), such as a mechanical chest compression device as illustrated in FIG. 1, may be utilized in conjunction with the first medical device 104(1) in order to assist with the monitoring and/or treatment of the subject. For example, the second medical device 104(2) may include any other medical device configured to detect a parameter of the subject 102, or administer a treatment (e.g., an electrical shock, pacing, chest compressions, etc.) to the subject.

In some examples, the medical device 104(1) may include an environmental sensor(s) 130 configured to detect an environmental parameter indicative of an environment surrounding the medical device 104(1), such as during a rescue event, during transport (e.g., movement of the medical device 104(1) to and from the rescue scene), during storage, or any combination thereof.

The environmental sensor may include a temperature sensor configured to measure a temperature of the environment external to the medical device 104(1). In some instances, environmental temperature may provide insight into how the medical device 104(1) functions and/or is used. In various examples, extreme temperatures (both hot and cold) may play a role in the performance of the medical device 104(1) and/or accessory devices stored with the medical device 104(1). For example, relatively high temperatures (e.g., over 35 degrees Celsius, such as in Texas) may degrade internal circuit components of the medical device 104(1), degrade an electrically conductive gel of the sensor electrodes 110 during storage, or cause other damage that may interfere with the function of the medical device 104(1). In some cases, relatively cold temperatures (e.g., under 0 degrees Celsius, such as in Alaska) may cause circuit components of the medical device 104(1) to break or may reduce their conductivity, which may also cause the medical device 104(1) to malfunction. These and similar problems, for instance, may not be apparent during the testing and development of the medical device 104(1) prior to deployment.

In some cases, the environmental sensor(s) 130 may include a humidity sensor (e.g., a hygrometer) configured to measure a humidity of the environment external to the medical device 104(1). In some instances, a humidity level of the environment may affect the performance of the medical device 104(1) and/or a condition of the subject 102 during the rescue event. Humidity, for instance, may promote corrosion of electronic components and/or circuitry in the medical device 104(1), which can lead to electrical shorts, intermittent connections, and/or permanent damage of components of the medical device 104(1). In some cases, humidity can impact the function of accessory devices. For example, humidity can impact the calibration and/or accuracy of a light-based sensor for detecting an amount of CO₂ in the airway of the subject 102. Thus, if the medical device 104(1) is operating in a relatively humid environment (e.g., Hawaii) may operate differently than if the medical device 104(1) is operating in a relatively dry environment (e.g., Arizona).

In various examples, environmental characteristics, such as temperature and/or humidity, may affect how a sensor functions. In some cases, environmental characteristics of the medical device 104(1) impact how effectively an adhesive adheres the sensor electrodes 110 and/or the defibrillation electrodes 128 to the subject 102. In some examples, environmental characteristics impact how effectively a suction cup of a chest compression device adheres to the subject 102. In some cases, environmental temperature may affect the performance and/or lifespan of batteries used in the medical device 104(1) (e.g., high temperatures can accelerate battery degradation and reduce capacity while low temperatures can decrease battery efficiency and cause voltage fluctuations). In some instances, sensor accuracy may be affected by changes in ambient temperatures (e.g., variations in accuracy or calibration due to changes in ambient temperatures, which affect reliability and precision of measurements of data collected by the medical device 104(1)). Rapid changes in temperature and/or humidity may cause ingress of moisture which can lead to material degradation of plastics, adhesives, seals, etc. which can lead to warping or deterioration of the medical device 104(1). In some instances, environmental data may be associated with a misuse of the medical device 104(1).

In some cases, the environmental sensor(s) 130 may include an accelerometer configured to detect movement of the medical device 104(1). The accelerometer data can be used to determine whether the medial device 104(1) has been dropped, whether the medical device 104(1) was being moved during treatment (e.g., during administration of a shock), whether the medical device 104(1) is transported between locations frequently as opposed to remaining in a particular geographical location (e.g., a medical device 104(1) that travels in an ambulance or police car versus a medical device 104(1) that remains in a hospital, school, hotel, etc.) and the like. In some instances, accelerometer sensor data can be used to determine that the medical device 104(1) and/or accessories associated with the medical device (e.g., defibrillation electrodes, pulse oximeter probe) was used in a moving vehicle or a moving stretcher/cot. One or more algorithms may be used to process the accelerometer sensor data and be used to determine, for example, to identify a segment of time (e.g., a start and end time) during use of the medical device when it was used in a moving vehicle, stretcher/cot, etc.

That is, analysis of data indicative of the environmental condition(s) may indicate that the medical device 104(1) was exposed to extreme conditions (e.g., improper storage) which can lead to unsafe operation. In some cases, the analysis of the data can identify types of environmental conditions that cause unsafe or unreliable operation of the medical device 104(1), or other similar medical devices. As such, gathering and analyzing environmental data may provide insight into how medical devices may be improved.

The first medical device 104(1) and/or the second medical device 104(2) may be configured to identify, generate and/or receive data including identifying data, parameter data and/or operational data. As used herein, “receiving data” may refer to the process of obtaining or acquiring data from an external source such as sensors, instruments, networks, or other devices. The received data may be raw data or preprocessed data and may be in various forms (e.g., digital signals, analog signals, text, images, audio, video, etc.). As used herein, “identifying data” may refer to recognizing or determining the characteristics, attributes, and/or patterns of the data received. Identification of data may include parsing, classifying, labeling, and/or categorizing the data based on a predefined criteria, parameter, or rule. The process of identifying data may assist in distinguishing relevant data from noise, extract meaningful features, and organize the data for analysis. In particular examples, the medical device 104(1) may be configured to annotate the data at the device level (e.g., aggregate anonymized data and automatically annotate it prior to exporting the annotated data to an external computing device(s)). As used herein, “data generating” may refer to the process of creating or producing new data based on existing information or through computational processes. Generating data can involve modeling, synthesis, annotating, and/or transformation of existing data into new forms or representations. For example, generating new data may include generating encrypted data by encrypting privileged data using an encryption algorithm and a unique encryption key. In some instances, the data may be grouped and/or clustered using various techniques and algorithms (e.g., using K-means clustering techniques, hierarchical clustering techniques, density-based spatial clustering techniques, gaussian mixture models, spectral clustering, agglomerative clustering, self-organizing maps (SOM) techniques, and the like).

In the case of a monitor-defibrillator, the monitor-defibrillator may receive, identify, and/or generate various data types. For example, the monitor-defibrillator may be configured to receive, identify, and/or generate identifying data including an identifier of the subject including information used to identify the subject or patient. An identifier of a subject may include a subject name, subject identification number (e.g., patient identification number, medical record number, health insurance number, social security number, etc.), sex, age, weight, medical history, physiological data, date of birth, address, phone number, biometric information (e.g., fingerprints, iris scans, facial recognition, etc.), and the like. The monitor-defibrillator may receive and/or generate data including an identifier of the user (e.g., the rescuer's name, electronic identification number, biometric data, medical device purchaser information (e.g., a hospital, organization, etc.) and the like. The monitor-defibrillator may be configured to generate, identify, and/or receive data representing a location of the monitor-defibrillator (e.g., geographic coordinates, address, elevation, etc.) using one or more sensor(s) and/or systems (e.g., GPS sensor, global navigation satellite system, Wi-Fi positioning system (WPS), cellular triangulation or WLAN, etc.) which may be indicative of the identity of the subject 102. The monitor-defibrillator may generate receive, identify, and/or generate data representing a time of a rescue event. The monitor-defibrillator may be configured to generate parameter data indicating an ECG of the subject and the physiological parameter of the subject. The monitor-defibrillator identifies operational data indicating an input signal and the electrical shock.

The medical device 104(1) may be configured to identify privileged data (or identifying data). Privileged data may include information that is sensitive, confidential, and/or is protected by laws and regulations to ensure patient privacy and confidentiality. In some instances, privileged data may include information that can be used to derive the identity of a particular subject and/or rescue event. In at least one example, the privileged data is stored in a record associated with the subject 102. For example, when treatment is initiated utilizing a monitor-defibrillator, a patient record or an event log may be generated to document details of treatment and/or monitoring of the subject 102 and the subject's 102 response to the treatment. The patient record may include one or more of a date, time, location (e.g., geographic region, country, state, address, geographic coordinates, elevation, humidity level and/or temperature of the environment during the event, etc.) when treatment is administered, patient identification, type of treatment (e.g., particular treatment administered, such as defibrillation, cardioversion, or monitoring parameters adjusted), response to treatment (e.g., observations or assessments of the subject's response to the treatment, changes in vital signs, heart rhythm, symptoms, etc.), information association with the user 106 (e.g., healthcare provider) involved in administering treatment, outcome or result of the treatment administered (e.g., whether the treatment was successful in restoring normal heart rhythm or stabilizing a condition of the patient), an adverse event (e.g., documentation of a complication associated with a treatment), and the like. In some instances, the patient record may include user generated annotations (e.g., progress notes, physical examination notes, and/or other observations made by a healthcare provider and entered into the patient record) and/or documentation related to the case or file associated with the subject (e.g., a patient's medical records including, for example, a medication list, procedural history, family history, diagnosis records, vaccination history, allergies, and the like).

Transmission and storage of various types of privileged data described herein may be regulated or prohibited in view of various laws, rules, and regulations. For instance, the European Union (EU) enforces the General Data Protection Regulation (GDPR), governing the transfer of personal data collected and/or stored within the borders of EU member states. In some cases, entities controlling devices within EU borders are legally obligated, under the GDPR, to prevent personal data from being transferred to devices outside of EU borders. Thus, it may be prohibited, in some cases, for the medical device 104(1) to transmit privileged data to an external device in violation of an applicable data privacy law that applies to the geographic region in which the medical device 104(1) or the external device is located. Nevertheless, other data generated by the medical device 104(1) may be highly pertinent to improving the medical device 104(1), other similar medical devices 104(1), or identifying important trends associated with the use of the medical device 104(1) within a population including the subject 102.

The medical device 104(1) may be configured to generate de-identified data 132 by anonymizing privileged data associated with the subject 102 and the event. De-identification (or anonymization) of data may include removing and/or obscuring identifying data such that patient privacy is protected while still enabling the data to be utilized for research, analysis, or other purposes. For example, a de-identification component of the medical device 104(1) may anonymize identifying data (e.g., by removing direct identifiers, such as names, addresses, medical identification numbers, and/or any other information that could directly identify a subject), pseudonymize identifying data (e.g., by replacing identifying information with artificial identifiers or pseudonyms such that the data is not directly linked to a real identity of a subject), generalize the identifying data (e.g., by generalizing or aggregating data to a broader level such as an age range), suppress the identifying data (e.g., by removing or suppressing a data field from a dataset that meets or exceeds a threshold), mask the data (e.g., by replacing particular data values with similar but fictitious values), tokenize the identifying data (e.g., by replacing the identifying data with randomly generalized tokens or codes that are utilized to represent the original data without revealing the identifying data), and the like. In some instances, a combination of de-identification techniques may be utilized to de-identify various data or data types. For instance, a first portion of the identifying data (e.g., a patient name) may be removed, and a second portion of the identifying data may be generalized (e.g., particular location of the event may be generalized to a larger geographic region such that a particular location of the event is not identifiable).

In some instances, a de-identification component of the medical device 104(1) may be configured to generate de-identified data 132 based at least in part on encrypting, via a processor, the privileged data using an encryption algorithm and a unique encryption key prior to transmitting the data to an external device. As used herein, the term “encrypt,” and its equivalents, refers to a process of translating data from one format (e.g., an unencoded format) into an encoded format. In various cases, the encoded format is referred to as “ciphertext.” Unencoded data, which has not been encrypted, may be referred to as being in “plaintext.” In various examples, a de-identification component associated with the medical device encrypts data using at least one encryption key. An encryption key is a parameter that defines the translation of data from the one format into the encoded format. As used herein, the term “decrypt,” and its equivalents, refers to a process of translating data from an encoded format into another format (e.g., an unencoded format), such as a plaintext format. In various examples, an entity encrypts data using at least one decryption key. A decryption key is a parameter that defines the translation of data from the encoded format into the other format. A link key, for example, is an encryption and/or decryption key.

Various cryptographic techniques can be utilized in accordance with the features described in this disclosure. For example, data can be encrypted and decrypted via a symmetric key, wherein the encryption key and the decryption key are equivalent. In some cases, data can be encrypted and decrypted via asymmetric keys, wherein the encryption key and the decryption key are different. Cryptographic hash functions (CHFs) are examples of cryptographic techniques. Examples of cryptographic techniques include the Data Encryption Standard (DES), Advanced Encryption Standard (AES), Elliptic Curve Cryptography (ECC), Rivest-Shamir-Adleman (RSA), Secure Hash Algorithm (SHA)-1, SHA-2, SHA-3, BLAKE, BLAKE2, BLAKE3, WHIRLPOOL, MD2, MD4, MD5, MD6, Temporal Key Integrity Protocol (TKIP), Rivest cipher 4 (RC4), variably modified permutation composition (VMPC), blowfish, Twofish, Threefish, Tiny Encryption Algorithm (TEA), Extended TEA (XTEA), Corrected Block TEA (XXTEA), Diffie-Hellman exchange (DHE), elliptic curve DHE, super-singular isogeny Diffie-Hellman (SIDH) key exchange, and so on. Any suitable encryption or decryption technique can be used in accordance with implementations of this disclosure.

The medical device 104(1) and medical device 104(2), in response to generating de-identifying data, may transmit the de-identified data 132 to an external computing device(s) 136 via a communication network(s) 134. The external computing device(s) 136 may include a remote server configured to receive the de-identified data, perform an analysis on the received de-identified data 132, and identify a trend associated with the de-identified data 132. As used herein, the term “trend,” and its equivalents, refers to a pattern, correlation between data points, or general direction in which data points are moving or changing over time or across different observations or events. For instance, a trend may indicate an overall direction or tendency of data points to increase, decrease, or remain stable over time or across observations or events. A trend may indicate long-term patterns or tendencies in the data rather than short-term fluctuations or noise. A trend may indicate systematic changes in an underlying process or phenomena being observed, rather than random variations. A trend may indicate quantitative and/or qualitative changes in the data (e.g., an increase or decrease in values as well as changes around a central value). A trend may indicate correlations between different data types that have not previously been identified. The analysis may provide insight into how a medical device, or a fleet of medical devices, is used or misused, how well it functions, and what improvements can or should be made to the medical device(s). For instance, a trend may indicate misuse of a particular medical device, a type of medical device, and/or a fleet of medical devices. For example, analysis of the data may reveal trends and changes in physiological parameters that occur gradually or abruptly, trends that indicate disease progression that was not previously known, trends that indicate responses to treatment, recovery from illness, or worsening of symptoms.

In some examples, a trend may indicate changes in physiological parameters associated with geographic location, climate variations, seasonal variations in response to environmental factors such as temperature, humidity, sunlight exposure, and allergen levels. For instance, multiple instances of a particular respiratory pattern in a de-identified geographic region (e.g., a particular city) may be indicative of an outbreak of a respiratory virus in that geographic region.

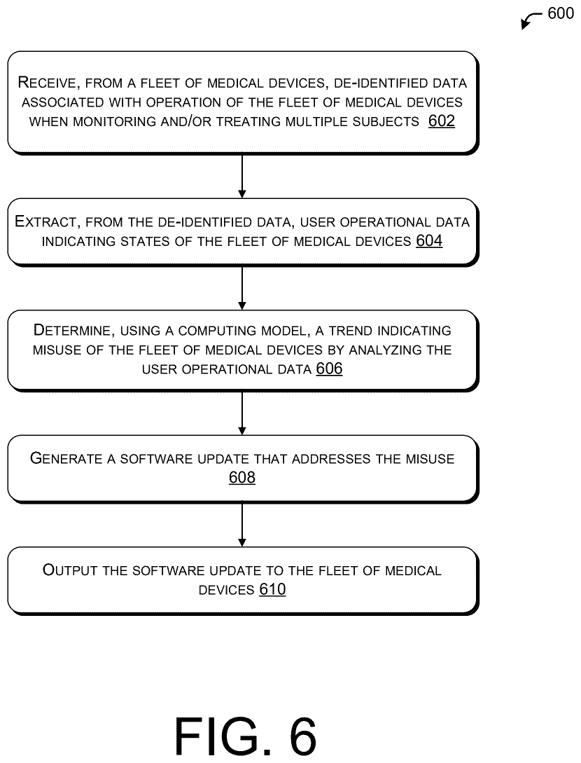

In some instances, the de-identified data 132 may be analyzed using a computing model (e.g., a machine learning model, statistical model, etc.). Example computing models are discussed below in relation to FIG. 6. The analysis may provide insight into odd events, such as whether excessive current is supplied to a subject (e.g., via a monitor defibrillator) which can lead to potential consequences such as tissue damage, cardiac injury, burns, damage to the device, systemic effects to the subject (e.g., cardiac arrest, neurological damage, multi-organ failure, etc.). The collected operational data provides insight into patterns or trends indicating how a user(s) interacts with the medical device and how the medical device operates in response to the user operational data. For example, the medical device may be configured to present a message, notification, or alert to the user 106 via a user interface (e.g., display 114) and may determine that the user responded to the alert in some particular manner (e.g., ignore the alert, change a setting associated with the device, respond after at threshold period of time, selected a user interface element, or any other response). Depending on the response to the alert, the external computing device may utilize one or more computing models to determine that users are not noticing the message, notification, or alert based at least in part on the type of alert, location of the alert on the user interface, etc. That is, particular user interface designs assume that users of medical devices look from left to right, or up to down, etc. depending on a region the medical device is used in. In this case, the issue is not a failing of the medical device(s), but rather a placement of the user interface elements or types of user interface elements.

As another example, a trend may indicate the detection of a new artifact associated with electrodes of a medical devices, ECG artifacts (or ECG noise), etc. so that appropriate corrective action is taken as needed. In some examples, a trend may provide insight into the accuracy of automated algorithm used to determine heart rhythm, heart rate, ECG interpretation etc. In some examples, a trend may identify an artifact due to device motion, patient motion, detachment of equipment (e.g., leads that are stretched or twisted, suction cups that are detached during use, a stretching or bending of a subject's skin under an electrode, etc.). A trend that identifies an artifact due to motion may be movement by the subject 102, respiration, movement of the subject 102 by a user (e.g., by a care provider during cardiopulmonary resuscitation), or transport motion (e.g., during ambulance transport). This information provides insight into improvements to medical devices, continuous post-market device performance monitoring, as well as spot systematic medical device issues earlier than when relying on user reports.

As an example, a computing model may analyze the de-identified data and identify the presence of an artifact in physiological parameter data received from medical devices being used in a particular geographic region, which may be indicative of broad misuse of medical device(s) in that particular geographic region. Broad misuse of medical device(s) in a particular geographic region may be due to a number of factors, such as lack of training or improper training, misunderstanding of instructions, lack of familiarity, fatigue or stress, overreliance on automation, equipment failure or inadequate maintenance of equipment, cultural or language barriers, etc. For instance, an artifact in data representative of a physiological parameter may indicate that the physiological parameter was detected by an incorrect sensor device (e.g., a sensor device designed for an adult used on a patient who is a child, a sensor device that is improperly connected to the medical device, etc.). In some examples, this can then be reported (e.g., in the form of an alert) to the medical devices in that geographic region. In some cases, a software update may be generated and output to appropriate medical device(s).

As another example, a computing model may analyze the de-identified data and identify a trend indicative of challenges with switching between various modes or settings. For instance, a display screen of a medical device may present various mode options or indications. The mode indications indicate what kind of filter or algorithms are active and/or displayed. In some examples, the mode indication presented is selectable, such that a user can activate and/or deactivate various modes (e.g., advisory mode, manual mode, adult mode, pediatric mode, etc.) by entering a user input signal (e.g., a touch signal received by one or more touch sensors corresponding to an area of the mode indication displayed on the display screen) associated with the mode indications. For example, a medical device with separate pediatric and adult modes, the button presses associated with switching between the two modes can be tracked to determine if the user interface requires improvement. For instance, the collected data may indicate that a user repeatedly presses pediatric mode, but the medical device does not switch modes that may indicate that the medical device needs to be capable of switching modes in more than one circumstance.

As another example, a computing model may analyze the de-identified data and identify a trend indicative of detection of mechanical chest compressor suction cup detachment events from a chest of a subject. That is, a combination of chest compressor force data during a decompression phase and the data from a CPR feedback puck and/or scene audio data can be analyzed using a computing model to assess the likelihood that the medical devices are being misused.

As another example, a computing model may analyze the de-identified data and identify a trend indicative of incompatible user settings between compatible devices and provide customer feedback for adjustment. For example, if audio CPR prompts for a monitor/defibrillator and a chest compression device are setup in a way that conflict with each other, it can be detected and the user informed. This is achieved through collection and analysis of data related to medical device ownership data, global positioning data, and/or medical device proximity data to other medical devices.

As another example, a computing model may analyze the de-identified data and track algorithm performance over time. For example, the de-identified data provides insight into how often a medical device prompts for a pause in chest compressions during CPR, the sensitivity and specificity of shock advisory algorithms or ST elevation myocardial infarction (STEMI) detection algorithms over time, etc. In some examples, de-identified data associated with artifact detectors and/or environmental sensors may be utilized in addition to the algorithm performance data in order to gain insight into when the algorithms tend to make incorrect decisions.

Analysis of the de-identified data may be used to track the performance and effectiveness of medical device alarms, errors, timeouts, etc. and the resulting user responses, such as silencing alarms, retaking measurements, and the like. This de-identified data may provide insight into how to improve related medical device design. For example, a computing model may identify a trend that indicates a medical device alert is not effective because of a detected delay of a user response exceeds a threshold response time (e.g., a user responded to an alert more than 15 seconds after an alert or error message was output to the user). In some instances. The computing model may identify an effectiveness of certain alert types (e.g., visual alert via display, aural alert, haptic alert, text, light indication via light emitters, an alarm, a voice, a buzz, etc.). For example, a trend may indicate that in some instances an aural alert is more effective at alerting a user (e.g., a rescuer) to sudden deteriorations in a condition of a subject than an alert displayed via a user interface, or an alert including light emitters is more effective than an aural alert on its own. These are merely examples and the collected data may provide insight into various other trends and correlations as discussed throughout the disclosure.

In response to identifying a trend, the external computing device(s) 136 may generate and output an alert 138. The alert 138 may be presented via a user interface associated with the external computing device(s) 136. The alert may be any type of alert or notification that informs a user of suggested device improvements and/or identifies information the user may be interested in knowing regarding performance of the medical devices.

In some examples, a user (e.g., a researcher) may request to view the non-anonymized data in whole or in part. For example, a researcher may want to access a particular record associated with a subject in a non-anonymized format or a portion of a particular record. In some examples, the non-anonymized data and its anonymized counterpart may be tagged with the same identifier and stored at least temporarily in a memory on the medical device and/or external memory (e.g., volatile memory (such as random access memory (RAM)), and/or non-volatile memory (such as read only memory (ROM), flash memory, etc.).

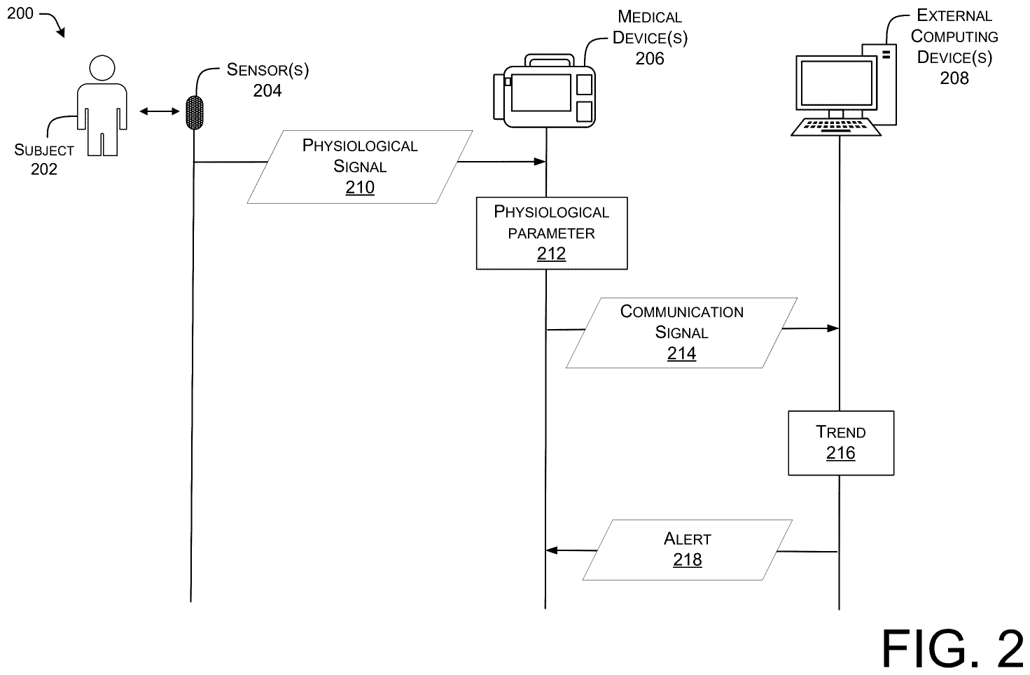

FIG. 2 illustrates example signaling 200 for facilitating communication between devices. In particular, the signaling 200 is between a sensor(s) 204, medical device(s) 206, and external computing device(s) 208. Various messages within the signaling 200 are transmitted over at least one wired connection and/or at least one wireless connection. Although not specifically illustrated, various messages within the signaling 200 are transmitted via one or more intermediary devices.

In examples, a sensor(s) 204 may be coupled to a subject 202 (e.g., a patient). The sensor 204 is configured to detect at least one physiological parameter of a subject. In various examples, the sensor 204(s) includes at least one of an electrode, a detection circuit, a flow sensor, an oxygen sensor, a carbon dioxide sensor, a non-invasive blood pressure (NIBP) sensor (e.g., a blood pressure cuff, an ultrasound-based blood pressure sensor, etc.), an oxygenation sensor (e.g., a regional oximetry sensor, a pulse oximetry sensor), or the like. In some implementations the sensor(s) 204 is coupled to the medical device 206.

The sensor(s) 204 generates a physiological signal 210 (e.g., an analog signal, electrical signal, etc.). For example, an analog signal obtained from the sensor(s) 204 is converted into a digital format suitable for processing and analysis by a medical device's digital electronics. A physiological signal 210 is a signal that is generated based on a biological process or activity within a body of a subject 202. A physiological signal 210 can include various types of data, such as electrical, chemical, or thermal signals, which reflect different physiological processes.

The physiological signal 210 is detected and received by a medical device(s) 206. The medical device(s) analyzes the physiological signal 210 in order to generate a physiological parameter 212 (e.g., a digital signal or data). A physiological parameter 212 can include, for example, vital signs (e.g., physiological parameters that reflect body functions such as body temperature, heart rate, blood pressure, respiratory rate, blood oxygenation (SpO₂)), cardiac parameters (e.g., parameters related to the cardiovascular system such as electrocardiogram (ECG/EKG), heart rhythm and heart rate variability, blood flow and cardiac output, etc.), respiratory parameters (e.g., end-tidal carbon dioxide (EtCO₂) levels, oxygen saturation, etc.), neurological parameters (e.g., electroencephalogram (EEG), intracranial pressure (ICP), neuromuscular function, etc.), metabolic parameters (e.g., blood glucose levels, blood pH, electrolyte levels, hormone levels, etc.), blood volume, blood flow, vascular resistance, and the like. In some instances, the medical device(s) 206 may preprocess the physiological signal 210 and to filter out noise, remove artifacts, and/or apply a signal processing algorithm to enhance a quality and/or accuracy of the physiological signal, such as frequency analysis, waveform analysis, or digital filtering techniques. The medical device(s) 206 de-identifies the data to ensure it is no longer protected personally identifiable information before transmitting to an external computing device.

In some examples, multiple medical devices may communicate with one another and exchange treatment parameters (e.g., monitoring device, treatment device, receiving device, etc.). As used herein, the term “treatment parameter” and its equivalents, refers to a characteristic of a treatment performed on a subject 202. In at least one example, a first device (e.g., a treatment device) may be a chest compression device that reports a frequency of chest compressions administered by the first device to a second device (e.g., a monitoring device or receiving device). A receiving device may perform one or more actions based on the physiological parameter 212 and/or treatment parameter. Actions performed by a monitoring device or a treatment device include initiating a measurement of a physiological parameter at a particular time or frequency, outputting a signal to a user, outputting a signal to the subject 202, performing a treatment at a particular time or frequency, adjusting a treatment parameter of an ongoing treatment, or any combination thereof. According to some examples, a monitoring device or a treatment device can instruct the other device to perform one or more actions. The receiving device, in turn, performs the action(s) based on the instruction from the monitoring device or the treatment device. For example, the monitoring device administers a defibrillation shock to the subject 202 at a particular time. By exchanging data or instructions, the monitoring device and the treatment device can coordinate monitoring and treatment of the subject 202.

To exchange data, multiple devices (e.g., a monitoring device and/or the treatment device) are configured to establish and/or communicate via a communication channel. As used herein, the term “communication channel,” and its equivalents, may refer to a medium over which a first endpoint (e.g., a sender) transmits information to one or more second endpoints (e.g., receivers). Examples of communication channels include wired connections, such as Ethernet or fiber optic paths, as well as wireless connections, such as Institute of Electronics and Electrical Engineers (IEEE) (e.g., WI-FI, BLUETOOTH, etc.) or 3^{rd} Generation Partnership Program (3GPP) (e.g., Long Term Evolution (LTE), New Radio (NR), etc.) connections. As used herein, the term “endpoint,” and its equivalents, may refer to an entity that is configured to transmit and/or receive data. Examples of endpoints include user equipment (UE) (e.g., mobile phones, tablet computers, etc.), computers, base stations, access points (APs), servers, compute nodes, medical devices, Internet of Things (IoT) devices, and the like.

In some implementations, the communication channel between multiple devices is established when the multiple devices are paired. In some examples, multiple devices may exchange one or more instructions, which can include an instruction to measure a physiological parameter, to begin a treatment, to end a treatment, a particular time or frequency at which to perform an action, an instruction to power on, an instruction to power off, an instruction to disconnect from a patient being treated or monitored, or a combination thereof. In particular cases, the multiple devices refrain from sharing substantive data (e.g., physiological parameters or other metrics, reports about the subject 202, instructions for treating the subject 202, etc.) until the devices are paired. As used herein, the term “paired,” and its equivalents, may refer to a state of multiple devices that have a shared link key that enables each device to cryptographically authenticate data it receives from any other device among the multiple devices. In some cases, paired devices communicate wirelessly.

The medical device(s) 206 transmits a communication signal 214 (e.g., an analog signal, cellular transmission, etc.) encoding the physiological parameter 212 to an external computing device(s) 208. The computing device(s) 206 analyzes the physiological parameter 212 as well as other received data (e.g., de-identified data representing a use of the medical device(s) 206) and identifies a trend 216. A trend may indicate misuse of a medical device(s) 206. In one example, analysis of the data may reveal trends and changes in physiological parameters that occur gradually or abruptly, trends that indicate disease progression that was not previously known, trends that indicate responses to treatment, recovery from illness, or worsening of symptoms. In some examples, a trend may indicate changes in physiological parameters due to geographic location, climate variations, seasonal variations in response to environmental factors such as temperature, humidity, sunlight exposure, and allergen levels.

The external computing device(s) 208 derives the physiological parameter by receiving the communication signal 214. The external computing device(s) 208 may receive communication signals from a fleet of medical devices. The communication signal 214 may include raw physiological data or preprocessed physiological data. In some examples, the external computing device(s) 208 may extract, from the de-identified data received from a fleet of medical device, patient data including physiological parameters of the multiple subjects or treatments administered to the multiple subjects. The external computing device 208 is configured to determine, using a computing model, a trend 216 indicating misuse of the fleet of medical devices.

In some instances, data transmitted from multiple medical devices is tagged with an identifier(s) (e.g., identification tags). Identifiers may be utilized to tag records that are associated with one another in order to facilitate storage and querying of records by type or category. For instance, data associated with different devices but a single patient and/or a single event, data associated with a single medical device, etc., may be tagged with a unique identifier. In a particular example, a defibrillator and mechanical chest compression device are applied to the same patient, and may each generate data that shares a single identifier associated with the patient. Data associated with the same identifier(s) may be stored in a database together (e.g., data transmitted from multiple medical devices from a single patient case can be tagged with identifiers to associate them together in the external computing device(s)).

In some examples, data generated, received, collected, and/or identified during an event may be stored in one or more databases. For example, data associated with separate medical devices may be stored at individual databases associated with that medical device. For instance, first data associated with a defibrillator (or other medical device) may be stored in a first database and second data associated with a mechanical chest compression device may be stored in a second database. In some examples, different data may be stored in different locations (e.g., databases) and the identifier (or identification tag) enables data from multiple, different locations to be retrieved so that it can be analyzed. By storing data associated with the same patient in different locations, the risk of a patient being identified by the data can be reduced.

In some examples, the external computing device(s) 208 outputs an alert 218 (e.g., another analog signal) to the medical device(s) 206 based at least in part on identifying the trend 216. The alert 218 may include an aural, visual alert, message, etc. In examples, the alert 218 may be associated with instructions to initiate a software update. In at least one example, in the case where a portion of the medical devices include chest compression devices, and the user operational data indicates suction cup detachment events, the alert may be associated with a software update that includes instructions to detect suction cup detachment events and, in response to detecting the suction cup detachment events, outputs an alert.

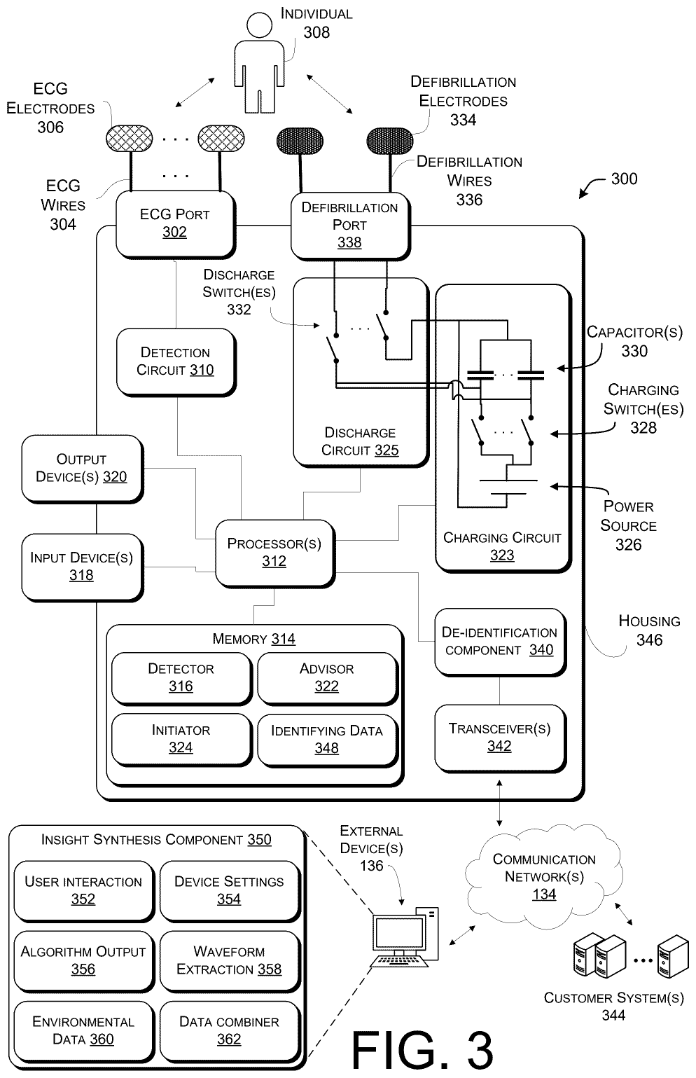

FIG. 3 illustrates an example external defibrillator 300 configured to perform various functions described herein. For example, the external defibrillator 300 is the monitor-defibrillator described above with reference to FIG. 1.

The external defibrillator 300 includes an electrocardiogram (ECG) port 302 connected to multiple ECG wires 304. In some cases, the ECG wires 304 are removeable from the ECG port 302. For instance, the ECG wires 304 are plugged into the ECG port 302 via connectors. The ECG wires 304 are connected to ECG electrodes 306, respectively. In various implementations, the ECG electrodes 306 are disposed on different locations on an individual 308. A detection circuit 310 is configured to detect relative voltages between the ECG electrodes 306. These voltages are indicative of the electrical activity of the heart of the individual 308.

In various implementations, the ECG electrodes 306 are in contact with the different locations on the skin of the individual 308. In some examples, a first one of the ECG electrodes 306 is placed on the skin between the heart and right arm of the individual 308, a second one of the ECG electrodes 306 is placed on the skin between the heart and left arm of the individual 308, and a third one of the ECG electrodes 306 is placed on the skin between the heart and a leg (either the left leg or the right leg) of the individual 308. In these examples, the detection circuit 310 is configured to measure the relative voltages between the first, second, and third ECG electrodes 306. Respective pairings of the ECG electrodes 306 are referred to as “leads,” and the voltages between the pairs of ECG electrodes 306 are known as “lead voltages.” In some examples, more than three ECG electrodes 306 are included, such that 5-lead or 12-lead ECG signals are detected by the detection circuit 310.

The detection circuit 310 includes at least one analog circuit, at least one digital circuit, or a combination thereof. The detection circuit 310 receives the analog electrical signals from the ECG electrodes 306, via the ECG port 302 and the ECG wires 304. In some cases, the detection circuit 310 includes one or more analog filters configured to filter noise and/or artifacts from the electrical signals. The detection circuit 310 includes an analog-to-digital (ADC) in various examples. The detection circuit 310 generates a digital signal indicative of the analog electrical signals from the ECG electrodes 306. This digital signal can be referred to as an “ECG signal” or an “ECG.”

In some cases, the detection circuit 310 further detects an electrical impedance between at least one pair of the ECG electrodes 306. For example, the detection circuit 310 includes, or otherwise controls, a power source that applies a known voltage (or current) across a pair of the ECG electrodes 306 and detects a resultant current (or voltage) between the pair of the ECG electrodes 306. The impedance is generated based on the applied signal (voltage or current) and the resultant signal (current or voltage). In various cases, the impedance corresponds to respiration of the individual 308, chest compressions performed on the individual 308, and other physiological states of the individual 308. In various examples, the detection circuit 310 includes one or more analog filters configured to filter noise and/or artifact from the resultant signal. The detection circuit 310 generates a digital signal indicative of the impedance using an ADC. This digital signal can be referred to as an “impedance signal” or an “impedance.”

The detection circuit 310 provides the ECG signal and/or the impedance signal one or more processors 312 in the external defibrillator 300. In some implementations, the processor(s) 312 includes a central processing unit (CPU), a graphics processing unit (GPU), both CPU and GPU, or other processing unit or component known in the art.

The processor(s) 312 is operably connected to memory 314. In various implementations, the memory 314 is volatile (such as random access memory (RAM)), non-volatile (such as read only memory (ROM), flash memory, etc.) or some combination of the two. The memory 314 stores instructions that, when executed by the processor(s) 312, causes the processor(s) 312 to perform various operations. In various examples, the memory 314 stores methods, threads, processes, applications, objects, modules, any other sort of executable instruction, or a combination thereof. In some cases, the memory 314 stores files, databases, or a combination thereof. In some examples, the memory 314 includes, but is not limited to, RAM, ROM, electrically erasable programmable read-only memory (EEPROM), flash memory, or any other memory technology. In some examples, the memory 314 includes one or more of CD-ROMs, digital versatile discs (DVDs), content-addressable memory (CAM), or other optical storage, magnetic cassettes, magnetic tape, magnetic disk storage or other magnetic storage devices, or any other medium which can be used to store the desired information and which can be accessed by the processor(s) 312 and/or the external defibrillator 300. In some cases, the memory 314 at least temporarily stores the ECG signal and/or the impedance signal.

The processor(s) 312 is operably connected to one or more input devices 318 and one or more output devices 320. Collectively, the input device(s) 318 and the output device(s) 320 function as an interface between a user and the defibrillator 300. The input device(s) 318 is configured to receive an input from a user and includes at least one of a keypad, a cursor control, a touch-sensitive display, a voice input device (e.g., a microphone), a haptic feedback device (e.g., a gyroscope), or any combination thereof. The output device(s) 320 includes at least one of a display, a speaker, a haptic output device, a printer, or any combination thereof. In various examples, the processor(s) 312 causes a display among the input device(s) 318 to visually output a waveform of the ECG signal and/or the impedance signal. In some implementations, the input device(s) 318 includes one or more touch sensors, the output device(s) 320 includes a display screen, and the touch sensor(s) are integrated with the display screen. Thus, in some cases, the external defibrillator 300 includes a touchscreen configured to receive user input signal(s) and visually output physiological parameters, such as the ECG signal and/or the impedance signal.

In various examples, the memory 314 includes a detector 316, which causes the processor(s) 312 to determine, based on the ECG signal and/or the impedance signal, whether the individual 308 is exhibiting a particular heart rhythm. For instance, the processor(s) 312 determines whether the individual 308 is experiencing a shockable rhythm that is treatable by defibrillation. Examples of shockable rhythms include ventricular fibrillation (VF) and ventricular tachycardia (V-Tach). In some examples, the processor(s) 312 determines whether any of a variety of different rhythms (e.g., asystole, sinus rhythm, atrial fibrillation (AF), etc.) are present in the ECG signal.

In some examples, the memory 314 includes an advisor 322, which, when executed by the processor(s) 312, causes the processor(s) 312 to generate advice and/or control the output device(s) 320 to output the advice to a user (e.g., a rescuer). In some examples, the processor(s) 312 provides, or causes the output device(s) 320 to provide, an instruction to perform CPR on the individual 308. In some cases, the processor(s) 312 evaluates, based on the ECG signal, the impedance signal, or other physiological parameters, CPR being performed on the individual 308 and causes the output device(s) 320 to provide feedback about the CPR in the instruction. According to some examples, the processor(s) 312, upon identifying that a shockable rhythm is present in the ECG signal, causes the output device(s) 320 to output an instruction and/or recommendation to administer a defibrillation shock to the individual 308.

The memory 314 also includes an initiator 324 which, when executed by the processor(s) 312, causes the processor(s) 312 to control other elements of the external defibrillator 300 in order to administer a defibrillation shock to the individual 308. In some examples, the processor(s) 312 executing the initiator 324 selectively causes the administration of the defibrillation shock based on determining that the individual 308 is exhibiting the shockable rhythm and/or based on an input from a user (received, e.g., by the input device(s) 318). In some cases, the processor(s) 312 causes the defibrillation shock to be output at a particular time, which is determined by the processor(s) 312 based on the ECG signal and/or the impedance signal.

The memory 314 also includes identifying data 348 (or privileged data). Identifying data may include information that is sensitive, confidential, and/or privileged data that is protected by laws and regulations. In some instances, privileged data may include information that identifies a particular subject and/or rescue event. In at least one example, the first data is stored in a record associated with the subject. For example, when treatment is initiated utilizing a monitor-defibrillator, a patient record or an event log is generated to document details of the monitoring and/or treatment of the subject and the subject's response. The patient record may include one or more of a date, time, location (e.g., geographic region, country, state, address, geographic coordinates, elevation, humidity level and/or temperature of the environment during the event, etc.), when treatment is administered, patient identification, type of treatment (e.g., particular treatment administered, such as defibrillation, cardioversion, or monitoring parameters adjusted), response to treatment (e.g., observations or assessments of the subject's response to the treatment, changes in vital signs, heart rhythm, symptoms, etc.), information associated with the user (e.g., healthcare provider) involved in administering treatment, outcome or result of the treatment administered (e.g., whether the treatment was successful in restoring normal heart rhythm or stabilizing a condition of the patient), an adverse event (e.g., documentation of a complication associated with a treatment), and the like.

The external defibrillator 300 includes a de-identification component 340 configured to generate de-identified data. De-identification of data may include removing and/or obscuring identifying data such that patient privacy is protected while still enabling the data to be utilized for research, analysis, or other purposes, as discussed above in relation in FIG. 1. For example, the de-identification component 340 may anonymize identifying data (e.g., by removing direct identifiers, such as names, addresses, medical identification numbers, and/or any other information that could directly identify a subject), pseudonymize identifying data (e.g., replace identifying information with artificial identifiers or pseudonyms such that the data is not directly linked to a subject's real identity), generalize the identifying data (e.g., generalize or aggregate data to a broader level such as an age range), suppress the identifying data (e.g., by removing or suppressing a data field from a dataset that meets a threshold), mask the data (e.g., by replacing particular data values with similar but fictitious values), tokenize the identifying data (e.g., by replacing the identifying data with randomly generalized tokens or codes that are utilized to represent the original data without revealing the identifying data), and the like. In some instances, a combination of de-identification techniques may be utilized to de-identify different types of data. In some instances, the de-identification component 340 can be configured to generate de-identified data based at least in part on encrypting, via a processor, the privileged data using an encryption algorithm and a unique encryption key prior to transmitting the data to an external device via transceiver 342.

The processor(s) 312 is operably connected to a charging circuit 323 and a discharge circuit 325. In various implementations, the charging circuit 323 includes a power source 326, one or more charging switches 328, and one or more capacitors 330. The power source 326 includes, for instance, a battery. The processor(s) 312 initiates a defibrillation shock by causing the power source 326 to charge at least one capacitor among the capacitor(s) 330. For example, the processor(s) 312 activates at least one of the charging switch(es) 328 in the charging circuit 323 to complete a first circuit connecting the power source 326 and the capacitor to be charged. Then, the processor(s) 312 causes the discharge circuit 325 to discharge energy stored in the charged capacitor across a pair of defibrillation electrodes 334, which are in contact with the individual 308. For example, the processor(s) 312 deactivates the charging switch(es) 328 completing the first circuit between the capacitor(s) 330 and the power source 326 and activates one or more discharge switches 332 completing a second circuit connecting the charged capacitor 330 and at least a portion of the individual 308 disposed between defibrillation electrodes 334.

The energy is discharged from the defibrillation electrodes 334 in the form of a defibrillation shock. For example, the defibrillation electrodes 334 are connected to the skin of the individual 308 and located at positions on different sides of the heart of the individual 308, such that the defibrillation shock is applied across the heart of the individual 308. The defibrillation shock, in various examples, depolarizes a significant number of heart cells in a short amount of time. The defibrillation shock, for example, interrupts the propagation of the shockable rhythm (e.g., VF or V-Tach) through the heart. In some examples, the defibrillation shock is 200 J or greater with a duration of about 0.015 seconds. In some cases, the defibrillation shock has a multiphasic (e.g., biphasic) waveform. The discharge switch(es) 332 are controlled by the processor(s) 312, for example. In various implementations, the defibrillation electrodes 334 are connected to defibrillation wires 336. The defibrillation wires 336 are connected to a defibrillation port 338, in implementations. According to various examples, the defibrillation wires 336 are removable from the defibrillation port 338. For example, the defibrillation wires 336 are plugged into the defibrillation port 338.

In various implementations, the processor(s) 312 is operably connected to one or more transceivers 342 that transmit and/or receive data over one or more communication networks 134. For example, the transceiver(s) 342 includes a network interface card (NIC), a network adapter, a local area network (LAN) adapter, or a physical, virtual, or logical address to connect to the various external devices and/or systems. In various examples, the transceiver(s) 342 includes any sort of wireless transceivers capable of engaging in wireless communication (e.g., radio frequency (RF) communication). For example, the communication network(s) 134 includes one or more wireless networks that include a 3rd Generation Partnership Project (3GPP) network, such as a Long Term Evolution (LTE) radio access network (RAN) (e.g., over one or more LTE bands), a New Radio (NR) RAN (e.g., over one or more NR bands), or a combination thereof. In some cases, the transceiver(s) 342 includes other wireless modems, such as a modem for engaging in WI-FI®, WIGIG®, WIMAX®, BLUETOOTH®, or infrared communication over the communication network(s) 134.

In various implementations, the external defibrillator 300 also includes a housing 346 that at least partially encloses other elements of the external defibrillator 300. For example, the housing 346 encloses the detection circuit 310, the processor(s) 312, the memory 314, the charging circuit 323, the de-identification component 340, the transceiver(s) 342, or any combination thereof. In some cases, the input device(s) 318 and output device(s) 320 extend from an interior space at least partially surrounded by the housing 346 through a wall of the housing 346. In various examples, the housing 346 acts as a barrier to moisture, electrical interference, and/or dust, thereby protecting various components in the external defibrillator 300 from damage.

In some implementations, the external defibrillator 300 is an automated external defibrillator (AED) operated by an untrained user (e.g., a bystander, layperson, etc.) and can be operated in an automatic mode. In automatic mode, the processor(s) 312 automatically identifies a rhythm in the ECG signal, makes a decision whether to administer a defibrillation shock, charges the capacitor(s) 330, discharges the capacitor(s) 330, or any combination thereof. In some cases, the processor(s) 312 controls the output device(s) 320 to output (e.g., display) a simplified user interface to the untrained user. For example, the processor(s) 312 refrains from causing the output device(s) 320 to display a waveform of the ECG signal and/or the impedance signal to the untrained user, in order to simplify operation of the external defibrillator 300.

In some examples, the external defibrillator 300 is a monitor-defibrillator utilized by a trained user (e.g., a clinician, an emergency responder, etc.) and can be operated in a manual mode or the automatic mode. When the external defibrillator 300 operates in manual mode, the processor(s) 312 cause the output device(s) 320 to display a variety of information that may be relevant to the trained user, such as waveforms indicating the ECG data and/or impedance data, notifications about detected heart rhythms, and the like.

The external defibrillator 300 is configured to transmit and/or receive data (e.g., ECG data, impedance data, data indicative of one or more detected heart rhythms of the individual 308, data indicative of one or more defibrillation shocks administered to the individual 308, etc.) with one or more external devices 136 via the communication network(s) 134. The external devices 136 include, for instance, mobile devices (e.g., mobile phones, smart watches, etc.), Internet of Things (IoT) devices, medical devices, computers (e.g., laptop devices, servers, etc.), or any other type of computing device configured to communicate over the communication network(s) 134. In some examples, the external device(s) 136 is located remotely from the external defibrillator 300, such as at a remote clinical environment (e.g., a hospital), research facility, etc. According to various implementations, the processor(s) 312 causes the transceiver(s) 342 to transmit data to the external device(s) 136 and/or a customer system(s) 344 (e.g., hospital(s) subscribed to a data service that provides the hospital access to the data). In some instances, the customer system(s) 344 may receive the de-identified data in an encrypted format as well as a decryption key that enables the customer system(s) 344 to decrypt the de-identified data. In some cases, the transceiver(s) 342 receives data from the external device(s) 136 and the transceiver(s) 342 provide the received data to the processor(s) 312 for further analysis.

The external devices(s) 136 include an insight synthesis component 350 where data from one or more medical devices is transmitted. The insight synthesis component 350 provides synthesis of insight into the use and performance of the medical device or fleet of medical devices as well as how the medical devices can be improved. The insight synthesis component 350 may be composed of several components including a user interaction 352 component, device settings 354 component, algorithm output 356 component, waveform extraction 358 component, environmental data 360 component, and/or a data combiner 362 component.

The user interaction 352 component is configured to extract, from the received de-identified data, information pertinent to user interactions with the medical devices such as button press events, voice commands, user entered data, selection of graphical user interface elements (e.g., user selected settings), mode selection (e.g., monitoring mode, pediatric/adult modes, pacing mode, configuration settings, etc.) alarm acknowledgement (e.g., if the device generates alarms or alerts, users may need to acknowledge or silence alarms by pressing specific buttons or interaction with the device interface), parameter adjustment (e.g., adjusting volume settings, display options, patient-specific settings), ECG lead selection (e.g., a user may select different lead configurations or views to visualize a subject's cardiac activity), defibrillation (e.g., timing associated with a user interacting with the device to deliver electrical shocks to the subject's heart at particular energy levels, etc.), changes to automatic device settings (e.g., a user adjusting or overriding an automatic pacing algorithms set by the device), navigation and menu selection, maintenance and self-tests initiated by a user, or any other user interactions with the medical device.

The device setting 354 component may be configured to extract, from the received de-identified data, information pertinent to the device settings and output. For example, device setting data may include device audio output, device display output (e.g., generated messages, alerts, notifications, etc.), default settings associated with the device, energy levels during defibrillation, battery levels over time, age of the device, ECG lead configuration during use, alarm thresholds (e.g., default alarm or notification thresholds for vital signs and physiological parameters, such as heart rate, blood pressure, oxygen saturation, respiratory rate, etc. may be configured to trigger audible and/or visual alerts when met or exceeded), communication and connectivity settings, language and display preferences (e.g., selecting language, protocols, display brightness, contract, connectivity options, etc.), default settings for arrhythmia detection and monitoring (e.g., detection thresholds, alarm priorities, etc.), default settings for ECG filtering and signal processing, and/or other device settings.

The algorithm output 356 component is configured to extract, from the de-identified data, algorithm outputs such as decisions from shock advisory algorithms (e.g., defibrillation algorithms that determine the timing, energy level, waveform morphology, and/or synchronization parameters for delivering shocks based on a patient's cardiac rhythm), QRS detection (representing the depolarization of ventricles), pacer spike detection (e.g., pacing algorithm that identifies the presence and timing of packing spikes within the ECG waveform), artifact rejection algorithms (e.g., algorithms that filter out noise, interference, and artifacts from physiological signals to improve signal quality and accuracy), alarm management algorithms (e.g., algorithms that monitor patient parameters and trigger alarms/alerts based on predefined thresholds or clinical rules), hemodynamic monitoring algorithms (e.g., algorithms that calculate cardiac output, stroke volume, systemic vascular resistance, and/or other hemodynamic parameters based on pressure waveform analysis or impedance measurements), respiratory monitoring algorithms (e.g., algorithms that are configured to analyze respiratory signals such airflow, respiratory rate, tidal volume, etc. and detect apnea, hypoventilation, respiratory distress, abnormal breathing patterns, etc.), and the like.

The waveform extraction 358 component is configured to extract, from the de-identified data received from a medical device or a fleet of medical devices, waveforms associated with particular events. For example, the waveform extraction 358 component may be configured to extract non-invasive blood pressure (NIBP) cuff data during NIBP acquisition, the ECG and impedance signals during rhythm analysis, and the like.

The environmental data 360 component may be configured to extract, from the de-identified data received from a medical device or a fleet of medical devices, data from environmental and location sensors. Environmental data may include, for example, ambient temperature data of the air surrounding the medical device during treatment, monitoring, and/or storage, relative humidity, air quality parameter, location data (e.g., geographic region, country, state, address, geographic coordinates, etc.), elevation data, and the like.

The data combiner 362 component is configured to combine data streams from multiple devices associated with a single event or patient and generate a single case from multiple devices and data sources. Data from multiple sources (e.g., multiple medical devices) may be extracted, transformed, and stored in a single data file

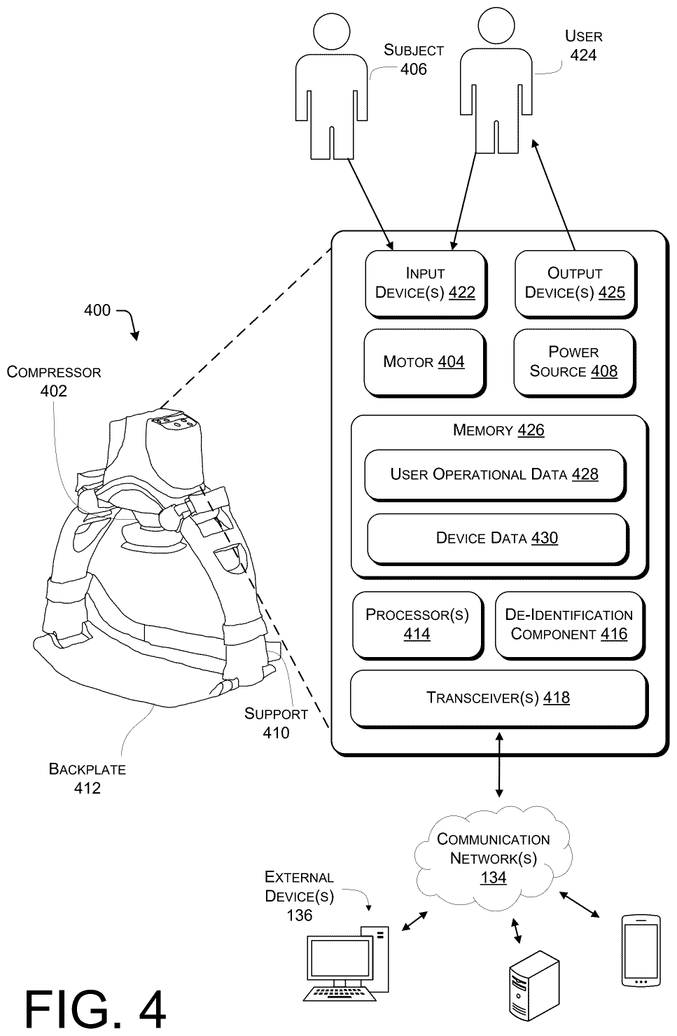

FIG. 4 illustrates a chest compression device 400 configured to perform various functions described herein. For example, the chest compression device 400 may be the medical device 104(2) described in FIG. 1.

In various implementations, the chest compression device 400 includes a compressor 402 that is operatively coupled to a motor 404. The compressor 402 physically administers a force to the chest of a subject 406 that compresses the chest of the subject 406. In some examples, the compressor 402 includes at least one piston that periodically moves between two positions (e.g., a compressed position and a release position) at a compression frequency. For example, when the piston is positioned on the chest of the subject 406, the piston compresses the chest when the piston is moved into the compressed position. A suction cup may be positioned on a tip of the piston, such that the suction cup contacts the chest of the subject 406 during operation. In various cases, the compressor 402 includes a band that periodically tightens to a first tension and loosens to a second tension at a compression frequency. For instance, when the band is disposed around the chest of the subject 406, the band compresses the chest when the band tightens.

The motor 404 is configured to convert electrical energy stored in a power source 408 into mechanical energy that moves and/or tightens the compressor 402, thereby causing the compressor 402 to administer the force to the chest of the subject 406. In various implementations, the power source 408 is portable. For instance, the power source 408 includes at least one rechargeable (e.g., lithium-ion) battery. In some cases, the power source 408 supplies electrical energy to one or more elements of the chest compression device 400 described herein.

In various cases, the chest compression device 400 includes a support 410 that is physically coupled to the compressor 402, such that the compressor 402 maintains a position relative to the subject 406 during operation. In some implementations, the support 410 is physically coupled to a backplate 412, cot, or other external structure with a fixed position relative to the subject 406. According to some cases, the support 410 is physically coupled to a portion of the subject 406, such as wrists of the subject 406.

The operation of the chest compression device 400 may be controlled by at least one processor 414. In various implementations, the motor 404 is communicatively coupled to the processor(s) 414. Specifically, the processor(s) 414 is configured to output a control signal to the motor 404 that causes the motor 404 to actuate the compressor 402. For instance, the motor 404 causes the compressor 402 to administer the compressions to the subject 406 based on the control signal. In some cases, the control signal indicates one or more treatment parameters of the compressions. Examples of treatment parameters include a frequency, timing, depth, force, position, velocity, and acceleration of the compressor 402 administering the compressions. According to various cases, the control signal causes the motor 404 to cease compressions.

In various implementations, the chest compression device 400 includes at least one transceiver 418 configured to communicate with at least one external device(s) 136 over one or more communication networks 134. The external device(s) 136, for example, may include at least one of a monitor-defibrillator, an AED, an ECMO device, a ventilation device, a patient monitor, a mobile phone, a server, or a computing device. In some examples, the external computing device(s) 136 may include a remote server configured to receive the de-identified data generated by the de-identification component 416, perform an analysis on the received de-identified data, and identify a trend associated with the de-identified data. In some implementations, the transceiver(s) 418 is configured to communicate with the external device(s) 136 by transmitting and/or receiving signals wirelessly. For example, the transceiver(s) 418 includes a NIC, a network adapter, a LAN adapter, or a physical, virtual, or logical address to connect to the various external devices and/or systems. In various examples, the transceiver(s) 418 includes any sort of wireless transceivers capable of engaging in wireless communication (e.g., RF communication). For example, the communication network(s) 134 includes one or more wireless networks that include a 3GPP network, such as an LTE RAN (e.g., over one or more LTE bands), an NR RAN (e.g., over one or more NR bands), or a combination thereof. In some cases, the transceiver(s) 418 includes other wireless modems, such as a modem for engaging in WI-FI®, WIGIG®, WIMAX®, BLUETOOTH®, or infrared communication over the communication network(s) 134. The signals, in various cases, encode data in the form of data packets, datagrams, or the like. In some cases, the signals are transmitted as compressions are being administered by the chest compression device 400 (e.g., for real-time feedback by the external device(s) 136), after compressions are administered by the chest compression device 400 (e.g., for post-event review at the external device 136), or a combination thereof.

In various cases, the processor(s) 414 generates the control signal based on data encoded in the signals received from an external device. For instance, the signals include an instruction to initiate the compressions, and the processor(s) 414 instructs the motor 404 to begin actuating the compressor 402 in accordance with the signals.

In some cases, the chest compression device 400 includes at least one input device 422. In various examples, the input device(s) 422 is configured to receive an input signal from a user 424, who may be a rescuer treating the subject 406. Examples of the input device(s) 422 include, for instance, at a keypad, a cursor control, a touch-sensitive display, a voice input device (e.g., a microphone), a haptic feedback device (e.g., a gyroscope), or any combination thereof. In various implementations, the processor(s) 414 generate the control signal based on the input signal. For instance, the processor(s) 414 generate the control signal to adjust a frequency of the compressions based on the chest compression device 400 detecting a selection by the user 424 of a user interface element displayed on a touchscreen or detecting the user 424 pressing a button integrated with an external housing of the chest compression device 400.

According to some examples, the input device(s) 422 include one or more sensors. The sensor(s), for example, is configured to detect a physiological parameter of the subject 406. In some implementations, the sensor(s) is configured to detect a state parameter of the chest compression device 400, such as a position of the compressor 402 with respect to the subject 406 or the backplate 412, a force administered by the compressor 402 on the subject 406, a force administered onto the backplate 412 by the body of the subject 406 during a compression, or the like. According to some implementations, the signals transmitted by the transceiver(s) 418 indicate the physiological parameter(s) and/or the state parameter(s).

The chest compression device 400 further includes at least one output device 425, in various implementations. Examples of the output device(s) 425 include, for instance, least one of a display (e.g., a projector, an LED screen, etc.), a speaker, a haptic output device, a printer, or any combination thereof. In some implementations, the output device(s) 425 include a screen configured to display various parameters detected by and/or reported to the chest compression device 400, a charge level of the power source 408, a timer indicating a time since compressions were initiated or paused, and other relevant information.

The chest compression device 400 further includes memory 426. In various implementations, the memory 426 is volatile (such as random access memory (RAM)), non-volatile (such as read only memory (ROM), flash memory, etc.) or some combination of the two. The memory 426 stores instructions that, when executed by the processor(s) 414, causes the processor(s) 414 to perform various operations. In various examples, the memory 426 stores methods, threads, processes, applications, objects, modules, any other sort of executable instruction, or a combination thereof. In some cases, the memory 426 stores files, databases, or a combination thereof. In some examples, the memory 426 includes, but is not limited to, RAM, ROM, EEPROM, flash memory, or any other memory technology. In some examples, the memory 426 includes one or more of CD-ROMs, DVDs, CAM, or other optical storage, magnetic cassettes, magnetic tape, magnetic disk storage or other magnetic storage devices, or any other medium which can be used to store the desired information. In various cases, the memory 426 stores instructions, programs, threads, objects, data, or any combination thereof, that cause the processor(s) 414 to perform various functions. In various cases, the memory 426 stores one or more parameters that are detected by the chest compression device 400 and/or reported to the chest compression device 400.

In implementations of the present disclosure, the memory 426 also stores user operational data 428 and/or device data 430. User operational data may include, for example, user input via the device keypad, cursor control, display, voice input, button presses, mode changes, etc., selection of a depth of chest compressions delivered by the device during cardiopulmonary resuscitation (CPR), a selected compression rate or frequency of chest compressions, a measurement of a compression force or pressure exerted by the chest compression device during compressions, changes in device settings or configurations during compressions, responses to alerts, messages or notifications output by the device, device maintenance and calibration procedures performed or initiated by a user, and any other information relating to a user's operation of the device.

Device data 420 may include, for example, output control signals generated by the chest compression device 400 during a rescue event (e.g., provided feedback to the user 424 related to the quality, depth, rate, timing, force, position, velocity, acceleration and/or effectiveness of the chest compressions as well as type of feedback provided including visual, auditory, and/or tactile), positioning of the suction cups, positions of the piston during compressions, a position of the compressor with respect to the subject during treatment, a force administered to the subject, the tension of the band, default device settings, configurations, and/or parameters (e.g., compression depth targets or compression rate thresholds), event logs and session summaries (e.g., time since compressions were initiated or paused, a number of compressions administered), device battery status and/or power consumption during the event, device maintenance and diagnostics data (e.g., self-checks and calibration procedures performed by the device itself) and any other relevant device data.

The chest compression device 400 further includes a de-identification component 416 configured to generate de-identified data. The de-identification component 416 may anonymize identifying data (e.g., by removing direct identifiers, such as names, addresses, medical identification numbers, and/or any other information that could directly identify a subject), pseudonymize identifying data (e.g., replace identifying information with artificial identifiers or pseudonyms such that the data is not directly linked to a subject's real identity), generalize the identifying data (e.g., generalize or aggregate data to a broader level such as an age range), suppress the identifying data (e.g., by removing or suppressing a data field from a dataset that meets a threshold), mask the data (e.g., by replacing particular data values with similar but fictitious values), tokenize the identifying data (e.g., by replacing the identifying data with randomly generalized tokens or codes that are utilized to represent the original data without revealing the identifying data), and the like. In some instances, a combination of de-identification techniques may be utilized to de-identify different types of data. In some instances, the de-identification component 416 can be configured to generate de-identified data based at least in part on encrypting, via processor 414, the privileged data using an encryption algorithm and a unique encryption key prior to transmitting the data to an external device via transceiver 418.

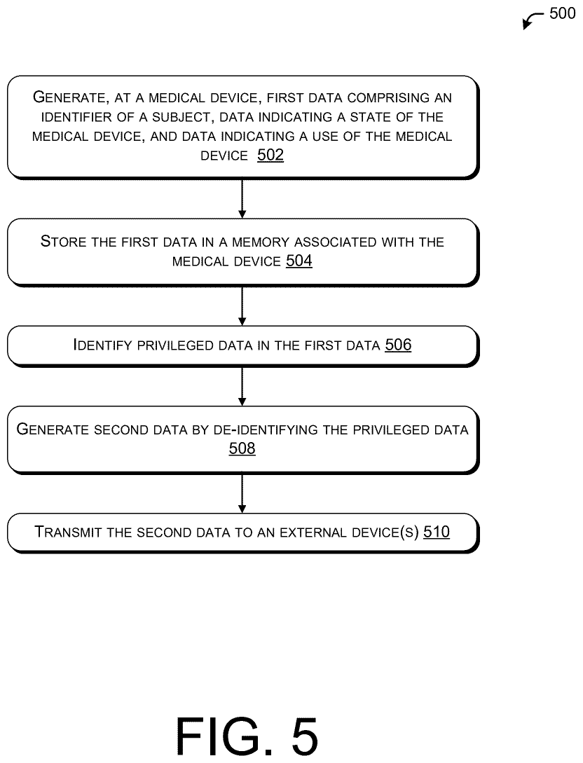

FIG. 5 illustrates an example process for de-identifying privileged data generated at a medical device and transmitting the de-identified data to an external device. The example process is performed by a medical device, such as a monitor-defibrillator, chest compression device, an automated external defibrillator (AED), patient monitor, etc.

At 502, the medical device generates, via a processor, first data including an identifier of a subject monitored or treated by the medical device, data indicating a state of the medical device during a rescue event, and data indicating a use of the medical device during the rescue event. An identifier of the subject may include information used to identify the subject or patient. For example, an identifier of the subject may include a subject name, subject identification number (e.g., patient identification number, medical record number, health insurance number, social security number, etc.), sex, age, weight, medical history, physiological data, date of birth, address, phone number, biometric information (e.g., fingerprints, iris scans, facial recognition, etc.), and the like. In some examples, the first data may include parameter data indicating an ECG of the subject, physiological parameter of the subject.

Data indicating a state of the medical device may include a model number, a serial number, a number of leads connected to the medical device, a type of lead connected to the medical device, a battery life, a volume level, a condition of one or more accessories associated with the medical device, physiological parameter data, shock advisory data, audio output data, display output data, or device motion during operation of the medical device. In some instances, data indicating a state of the medical device may include data that indicates the medical device is connected to another device (e.g., via wired or wireless communication, cloud-based communication, etc.). To provide a non-limiting example, during a rescue event, a mechanical chest compression device may be connected to or paired (e.g., via BLUETOOTH™) to a monitor/defibrillator device. These devices may include settings and features that are designed to be used in tandem when the devices are connected or paired. In some examples, data indicating the state of the medical device may include pairing data indicating information that was exchanged between two or more devices to establish a secure connection. The pairing data can be used to authenticate, authorize, and configure the two or more devices for communication. For example, BLUETOOTH™ pairing data may include the device name(s), MAC address, PIN code/passkey, encryption keys, Link key, etc., wi-fi pairing data may include a service-set identifier (SSID), encryption type (e.g., WPA2, WPA3, etc.), IP Address, and the like.

Data indicating use of the medical device may include data input by a user (e.g., patient data input by a user), a user response to an alert generated by the medical device (e.g., a user may silence an alarm output by the medical device), a time at which the user response was detected, a user response to a prompt generated by the medical device (e.g., the medical device may prompt the user to initiate treatment), an indication of a selection of one or more user interface elements, buttons, etc. of the medical device, and the like. In some instances, use of the medical device may include data indicating whether the medical device was dropped, whether the medical device was being moved during treatment (e.g., during administration of a shock), whether the medical device was shaking during treatment or use, and the like. For example, the medical device may include one or more of an accelerometer sensor, gyroscope sensor, magnetometer sensor, linear acceleration sensor, rotation vector sensor, etc. that enable the medical device to gather data related to whether the medical device is shaking or being moved during use. To provide one non-limiting example, a medical device may include an accelerometer that measures the acceleration forces acting on the medical device in three dimensions (e.g., x, y, and z axes) and can detect movement of the device (e.g., whether the medical device was being shaken, tilted, or was dropped).

In some examples, the first data may include operational data indicating an input signal and an electrical shock. In some instances, the first data may include a location of the medical device, a time of the rescue event, and/or an identifier of the user and/or subject.

At 504, the medical device stores the first data in a memory associated with the medical device. In some instances, the first data may be stored in the memory for a period of time (e.g., 24 hours, 7 days, 30 days, etc.).

At 506, the medical device identifies privileged data in the first data. Privileged data may include information that is sensitive, confidential, and/or is protected by laws and regulations to ensure patient privacy and confidentiality. In some instances, privileged data may include information that identifies a particular subject. In at least one example, the first data is stored in a record associated with the subject. For example, when treatment is initiated using a monitor-defibrillator. A patient record or an event log is generated to document details of the treatment of the subject and the subject's response. The patient record may include one or more of a date, time, location (e.g., geographic region, country, state, address, geographic coordinates, elevation, humidity level and/or temperature of the environment during the event, etc.) when treatment is administered, patient identification, type of treatment (e.g., particular treatment administered, such as defibrillation, cardioversion, or monitoring parameters adjusted), response to treatment (e.g., observations or assessments of the subject's response to the treatment, changes in vital signs, heart rhythm, symptoms, etc.), information association with the user (e.g., healthcare provider) involved in administering treatment, outcome or result of the treatment administered (e.g., whether the treatment was successful in restoring normal heart rhythm or stabilizing a condition of the patient), an adverse event (e.g., documentation of a complication associated with a treatment), and the like.

In some examples, the medical device may analyze the first data prior to or after de-identification of the first data and identify a correlation between the data indicating the state of the medical device and the data indicating the use of the medical device during the rescue event. For example, the first data may include data indicating an input signal from a user, wherein the input signal from the user includes data input by the user during the event, a user response to an alert generated by the medical device (e.g., whether the alert is silenced, a time delay between the output alert and a user response, whether the alert is ignored, etc.), a time at which the user response was detected, a user response to a prompt generated by the medical device, or an indication of selection of one or more user interface elements of the medical device. The first data may include a state of the medical device which may represent a model number, a serial number, a number of leads connected to the medical device, a type of lead connected to the medical device, a battery life, a volume level, a condition of one or more accessories associated with the medical device, physiological parameter data, measurement reliability data, shock advisory data, audio output data, display output data (e.g., what information is displayed to the user), prompt data (e.g. prompts generated by the medical device during treatment), alarm data, error data, failed acquisition data, or device motion during operation. In some examples, the medical device may determine a correlation between an environmental parameter indicative of the environment surrounding the medical device during use of the medical device (e.g., a temperature, humidity, air quality, noise level, atmospheric pressure, altitude, and the like) and a state of the medical device and/or a use of the medical device. The identified correlation may then be transmitted to an external device.

At 508, the medical device generates second data by de-identifying the privileged data. De-identification of data may include removing and/or obscuring identifying data such that patient privacy is protected while still enabling the data to be utilized for research, analysis, or other purposes. For example, a de-identification component of the medical device may anonymize identifying data (e.g., by removing direct identifiers, such as names, addresses, medical identification numbers, and/or any other information that could directly identify a subject), pseudonymize identifying data (e.g., replace identifying information with artificial identifiers or pseudonyms such that the data is not directly linked to a subject's real identity), generalize the identifying data (e.g., generalize or aggregate data to a broader level such as an age range), suppress the identifying data (e.g., by removing or suppressing a data field from a dataset that meets a threshold), mask the data (e.g., by replacing particular data values with similar but fictitious values), tokenize the identifying data (e.g., by replacing the identifying data with randomly generalized tokens or codes that are utilized to represent the original data without revealing the identifying data), and the like. In some instances, a combination of de-identification techniques may be utilized to de-identify various data. For instance, a first portion of the identifying data (e.g., a patient name) may be removed, and a second portion of the identifying data may be generalized (e.g., particular location of the event may be generalized to a larger geographic region).

In some instances, a de-identification component of the medical device may be configured to generate the second data based at least in part by encrypting, via a processor, the privileged data using an encryption algorithm and a unique encryption key prior to transmitting the data to an external device. In various examples, a de-identification component associated with the medical device encrypts data using at least one encryption key. An encryption key is a parameter that defines the translation of data from the one format into the encoded format. Various cryptographic techniques can be utilized in accordance with the features described in this disclosure. For example, data can be encrypted and decrypted via a symmetric key, wherein the encryption key and the decryption key are equivalent. In some cases, data can be encrypted and decrypted via asymmetric keys, wherein the encryption key and the decryption key are different.

At step 510, the medical device transmits the second data to an external device. In some instances, the medical device may generate and transmit the second data in response to an event (e.g., after a threshold period of time, in response to collecting a threshold amount of first data, in response to detecting an end of a rescue event, etc.). In some examples, the external device may be configured to restore the original data by decrypting the encrypted data. In some cases, the medical device may transmit, by a transceiver, the first data and the unique encryption key generated to a client database authorized to access the first data (e.g., hospital, healthcare provider, health insurance company, agency, research institution, legal or law enforcement entity, etc.).

FIG. 6 illustrates an example process 600 for determining a trend indicating misuse of a fleet of medical devices by analyzing data received from the fleet of medical devices. The process 600 is performed by at least one computing device (e.g., external computing device 136, remote server, etc.) and at least one processor.

At 602, the computing device may receive, from a fleet of medical devices, de-identified data associated with operation of the fleet of medical devices when monitoring and/or treating multiple subjects, the de-identified data omitting identifying information associated with multiple subjects. That is, the data is de-identified such that it no longer contains protected personally identifiable information.

At 604, the computing device may extract, from the de-identified data, user operational data indicating use of the fleet of medical devices and state(s) of the medical device during use, and/or any other relevant data generated during use of the fleet of medical devices. In some examples, the computing device may include an insight synthesis component configured to extract various data from the de-identified data and provide insight into the use and performance of a medical device or combination of medical devices. For example, the insight synthesis component may include a user interaction component configured to extract data pertinent to user interactions with a medical device, sch as button press events, audio commands provided by the user, user response to device display output, etc. An algorithm output component may be configured to extract algorithm output such as decisions from shock advisory algorithms, QRS and pacer spike detection, breath detection, etc. A wavefront extraction component may be configured to extract waveforms associated with particular events, such as an non-invasive blood pressure (NIBP) cuff pressure during NIBP acquisition, the ECG and impedance signals during rhythm analysis, etc. A device settings component may be configured to extract device settings and/or user selected settings, user entered data, environmental information and location sensor data, etc. The insight analysis component may be configured to combine data streams from multiple medical devices and/or data sources associated with a single event or subject to generate a single case from multiple medical devices and/or data sources. Data extracted by the insight synthesis component is synthesized into insights into how the medical devices are being used as well as insights into how the medical devices may be improved. Additionally, the extracted data may provide insight into the accuracy of algorithm outputs and user responses to algorithm outputs.

At 606, the computing device may determine, using a computing model, a trend (e.g., indicating how medical devices(s) are being used, detecting a novel medical condition connected to a particular region of the body, etc.). For example, the collected data may provide insight into misuse of a fleet of medical devices by analyzing the user operational data, treatment data, medical device state data, etc. For example, a trend may indicate a misuse of the medical devices due to delays between treatment recommendations output by the medical devices and treatments administered to the subjects, or nonindicated treatments administered to the subjects. In some examples, a computing model may be utilized to detect a failure in a user interface design. For instance, a user interface design may assume that users look from left to right, or up to down. However, users in different geographic regions may be inclined to view the user interface in a different manner. Trends indicating different use of medical device(s) may be identified.

In further examples, the collected data may provide insight into power needs of medical devices, therapy needs, and/or monitoring needs of medical devices by using aggregate medical device data related to frequency and duration of use for features such as therapy delivery, patient monitoring, and overall use and help guide development and improvement of features in medical devices including which features may be retired due to lack of use, malfunctioning, errors, etc.

In some examples, the computing model may include a machine learning model configured to receive user operational data, medical device state data, and environmental data as input and output a trend indicating a misuse of the fleet of medical devices. The computing model may include an artificial neural network including various layers that respectively process input data. For instance, an artificial neural network can include an input layer, one or more hidden layers, and an output layer. The input layer performs a pre-processing operation on the input data. The hidden layer(s) may perform various processing operations on the output from the input layer. The output layer, in various cases, processes the output from hidden layers(s). Each layer, in some cases, includes one or more nodes, which are defined by individual operations. In various cases, the hidden layer(s) include nodes that are connected to each other in parallel and/or series. Examples of artificial neural networks include feedforward neural networks, multi-layer perceptrons (MLPs), convolutional neural networks (CNNs), and backpropagation models. In various implementations, the operations performed by the layers and/or nodes within an artificial neural network included in the computing model is defined according to the parameters (e.g., weights, thresholds, filters, kernels, or other data objects that are utilized to perform operations of the computing model).

In some implementations, the computing model includes a nearest-neighbor model. One example of a nearest-neighbor model includes a k-nearest neighbor model. For example, a nearest-neighbor model defines various “neighbors,” which are points within a feature space, with associated class labels. When a new data point is mapped to the feature space, the new data point is classified based on the proximity (e.g., Euclidian distance, Manhattan distance, Minkowski distance, etc.) of its “neighbors” to the new data point as well as their associated classes. In some cases, the new data point is classified as belonging to a particular class if greater than a threshold number of neighbors within a threshold distance of the new data point are members of the class.

In various cases, the computing model may include a regression analysis model. The regression analysis model, for example, is defined by a regression function that defines relationships between one or more independent variables and one or more dependent variables. The regression function may further define one or more unknown parameters that define a relationship between the independent and dependent variables. In various implementations, the unknown parameters and/or the type of regression function (e.g., linear, quadratic, etc.), is defined according to the parameters (e.g., weights, thresholds, filters, kernels, or other data objects that are utilized to perform operations of the computing model).

In some cases, the computing model includes a clustering model. In various cases, a clustering model maps various data points (e.g., user operational data, device state data, etc.) to a feature space. Based on the proximity of groups of those data points in the features pace, one or more “clusters” are defined. An additional data point may be classified according to one or more of the clusters based on its proximity to the clusters (e.g., a center of the clusters, a boundary of the cluster, etc.). Examples of clustering models include k-means clustering, mean-shift clustering, expectation-maximization (EM) clustering, and agglomerative hierarchical clustering. The parameter(s), for example, include a threshold proximity within which a new data point is classified within a cluster, a density of points used to define a cluster, and the like.

In various examples, the computing model includes a principal component analysis model. In various implementations, a principal component analysis defines a collection principal components of unit vectors within a coordinate space based on a dataset (e.g., training dataset, user operational dataset, device state dataset, medical device battery life dataset, subject monitoring dataset, medical device mode switching dataset, algorithm performance dataset, alarm dataset, error dataset, etc.). The computing model, for example, is an orthogonal linear transformation of the dataset. Various weights of the model, for example, are included in the parameter(s).

The computing model may include a gradient boosting model. For example, the gradient boosting model is defined as a collection of prediction models (e.g., decision trees) that iteratively classify observed data. In various cases, the type of prediction model, weights in the prediction models, and the like, are defined by the parameter(s).

The computing model may include a random forest. The random forest, for instance, includes multiple decision trees that classify data in an ensemble fashion. In various implementations, the decision trees are defined by the parameter(s).

The collected data may provide insight into separate test therapies from actual patient therapies. That is, a combination of medical device data (e.g., ECG impedance, shock data, etc.) with environmental data (e.g., ambient sounds during use of the medical device, speak recognition, medical device motion during use, etc.) can be combined to more confidently separate test shocks (typically performed by hospital biomedical engineers) from actual patient therapies.

The collected data may be utilized to detect challenges with switching to between various modes or settings. For instance, a display screen of a medical device may present various mode options or indications. The mode indications indicate what kind of filter or algorithms are active and/or displayed. In some examples, the mode indication presented is selectable, such that a user can activate and/or deactivate various modes (e.g., advisory mode, manual mode, adult mode, pediatric mode, etc.) by entering a user input signal (e.g., a touch signal received by one or more touch sensors corresponding to an area of the mode indication displayed on the display screen) associated with the mode indications. For example, a medical device with separate pediatric and adult modes, the button presses associated with switching between the two modes can be tracked to determine if the user interface requires improvement. For instance, the collected data may indicate that a user repeatedly presses pediatric mode, but the medical device does not switch modes that may indicate that the medical device needs to be capable of switching modes in more than one circumstance.

The collected data may provide insight into detection of mechanical chest compressor suction cup detachment from a chest of the subject. This can be indicative of the use of a CPR feedback puck underneath the chest compressor. A combination of chest compressor force data during a decompression phase and the data from a CPR feedback puck and/or scene audio data can be used to assess the likelihood that the medical devices are being misused.

The collected data may provide insight into incompatible user settings between compatible devices and provide customer feedback for adjustment. For example, if audio CPR prompts for a monitor/defibrillator and a chest compression device are setup in a way that conflict with each other, it can be detected and the user informed. This is achieved through collection and analysis of data related to medical device ownership, global positioning data, and/or medical device proximity data to other medical devices.

The collected data may be utilized to track algorithm performance over time. For example, the collected data provides insight into how often a medical device prompts for a pause, the sensitivity and specificity of shock advisory algorithms or ST elevation myocardial infarction (STEMI) detection algorithms over time, etc. In some examples, data collected from artifact detectors and/or environmental sensors may be utilized in addition the algorithm performance data in order to gain insight into when the algorithms tend to make incorrect decisions.

The collected data may be utilized to track the performance and effectiveness of medical device alarms, errors, timeouts, etc. and the resulting user responses, such as silencing alarms, retaking measurements, and the like. This collected data may provide insight into how to improve related medical device design. For example, the computing model may identify a trend that indicates a medical device alert is not effective because of a detected delay of a user response exceeds a threshold response time (e.g., a user responded to an alert more than 15 seconds after an alert or error message was output to the user). In some instances. The computing model may identify that an effectiveness of certain alert types (e.g., visual alert via display, aural alert, haptic alert, text, light indication via light emitters, an alarm, a voice, a buzz, etc.). For example, a trend may indicate that in some instances an aural alert is more effective at alerting a user (e.g., a rescuer) to sudden deteriorations in a condition of a subject than an alert displayed via a user interface, or an alert including light emitters is more effective than an aural alert on its own. These are merely examples and the collected data may provide insight into various other trends and correlations.

At 608, the computing device generates a software update that addresses the misuse of the medical device(s). In the case where the fleet of medical devices include chest compression devices, and the user operational data input into the computing model, the software update may include instructions to detect suction cup detachment events and, in response to detecting the suction cup detachment events, output an alert. In some examples, the computing device may generate a recommendation for algorithm improvements as well as recommendations for user guidance.

At 610, the computing device outputs the software update to the fleet of medical devices. In some instances, prior to installing the software update, a user may be prompted to read and accept the software update prior to initiating install. In at least one example, the fleet of medical devices may be automatically updated. In examples, outputting the software update to the fleet of medical devices includes outputting a new alert in response to detecting an event (e.g., a new therapy need or monitoring need of a subject, detecting a challenge in switching between modes, detecting incompatible user settings, etc.), changing a location, size, type, or message of a visual alert on a screen of a medical device, changing a volume or tone of an auditory alert output by a speaker of the medical device, or refraining from outputting an existing alert.

### Example Clauses

1. A system, including: a monitor-defibrillator including: a monitoring circuit configured to detect an electrocardiogram (ECG) of a subject during a rescue event; a parameter sensor configured to detect a physiological parameter of the subject; a display configured to output a visual representation of the ECG and a visual representation of the physiological parameter; an input device configured to receive an input signal from a user; a treatment circuit configured to output an electrical shock to a heart of the subject in response to the input device receiving the input signal from the user; memory; a transceiver; and a processor configured to: generate first data including: identifying data including an identifier of the subject, a location of the monitor-defibrillator, a time of the rescue event, or an identifier of the user; parameter data indicating the ECG of the subject and the physiological parameter of the subject; and operational data indicating the input signal and the electrical shock; cause the memory to store the first data in a record associated with the subject; generate second data by de-identifying the first data, the second data including the parameter data and the operational data and omitting the identifying data; and cause the transceiver to transmit the second data; and a computing device configured to: receive the second data; receive third data from multiple medical devices, the third data being de-identified; identify a trend associated with the second data and the third data; and in response to identifying the trend, output an alert.
2. The system of clause 1, wherein the monitor-defibrillator further including an environmental sensor configured to detect an environmental parameter indicative of an environment surrounding the monitor-defibrillator during the rescue event, the environmental sensor including: a temperature sensor configured to measure a temperature of the environment external to the monitor-defibrillator; or a humidity sensor configured to measure a humidity of the environment external to the monitor-defibrillator.
3. The system of clause 1 or 2, wherein de-identifying the first data includes: identifying, via the processor associated with the monitor-defibrillator, privileged data; and generating encrypted data by encrypting, via the processor, the privileged data using an encryption algorithm and a unique encryption key, and wherein the second data includes the encrypted data.
4. A medical device, including: a physiological sensor configured to detect a physiological parameter of a subject during a rescue event; an environmental sensor configured to detect an environmental parameter indicating a state of the environment surrounding the medical device during the rescue event; and an input device configured to detect an input signal from a user; memory; and a processor configured to: generate first data including: an identifier of the subject or the rescue event; and data indicating the physiological parameter of the subject, data indicating the environmental parameter of the medical device, or data indicating the input signal from the user; cause the memory to store the first data; generate second data by de-identifying the first data, the second data omitting the identifier of the subject or the rescue event; and output the second data to an external device configured to: receive de-identified data from a fleet of medical devices; process the de-identified data; and identify a trend associated with the de-identified data.
5. The medical device of clause 4, wherein the environmental sensor includes: a temperature sensor configured to measure a temperature of an environment external to the medical device; or a humidity sensor configured to measure a humidity of the environment external to the medical device.
6. The medical device of clause 4 or 5, wherein the input signal from the user includes data input by the user, a user response to an alert generated by the medical device, a time at which the user response was detected, a user response to a prompt generated by the medical device, or an indication of a selection of one or more user interface elements of the medical device.
7. The medical device of clauses 4 to 6, wherein the identifier of the subject includes a subject name, subject identification number, sex, age, weight, medical history, or physiological data.
8. The medical device of clauses 4 to 7, wherein the first data further includes a state of the medical device, the state including a model number, a serial number, a number of leads connected to the medical device, a type of lead connected to the medical device, a battery life, a volume level, a condition of one or more accessories associated with the medical device, data indicating the medical device is paired to a separate medical device, physiological parameter data, measurement reliability data, shock advisory data, audio output data, display output data, prompt data, alarm data, error data, failed acquisition data, or device motion during operation.
9. The medical device of clauses 4 to 8, wherein de-identifying the first data includes: identifying, via the processor associated with the medical device, privileged data in the identifier; and generating encrypted data by encrypting, via the processor, the privileged data using an encryption algorithm and a unique encryption key, and wherein the second data includes the encrypted data.
10. The medical device of clauses 4 to 9, wherein the data is stored in a record associated with the subject.
11. The medical device of clauses 4 to 10, further including: a transceiver configured to transmit the first data to a client device operated by an authorized user.
12. The medical device of clauses 4 to 11, wherein the medical device includes a monitor-defibrillator, an automated external defibrillator (AED), a mechanical chest compression device, or a patient monitor.
13. A method performed by a medical device, the method including: generating, at the medical device, first data including: an identifier of a subject being monitored or treated by the medical device; data indicating a state of the medical device during a rescue event; and data indicating a use of the medical device during the rescue event; storing the first data in a memory associated with the medical device; generating second data by de-identifying, by a processor associated with the medical device, the first data; and transmitting the second data to an external device configured to: receive de-identified data from a fleet of medical devices; and identify a trend associated with the de-identified data.
14. The method of clause 13, wherein the identifier includes a subject name, subject identification number, sex, age, weight, medical history, or physiological data.
15. The method of clause 13 or 14, wherein the data indicating the state of the medical device includes a model number, a serial number, a number of leads connected to the medical device, a type of lead connected to the medical device, a battery life, a volume level, a condition of one or more accessories associated with the medical device, physiological parameter data, shock advisory data, audio output data, display output data, or device motion during operation.
16. The method of clauses 13 to 15, wherein the data indicating the use of the medical device includes data input by a user, a user response to an alert generated by the medical device, a time at which the user response was detected, a user response to a prompt generated by the medical device, or an indication of a selection of one or more user interface elements of the medical device.
17. The method of clauses 13 to 16, wherein de-identifying the first data includes: identifying, via the processor associated with the medical device, privileged data in the identifier; and generating encrypted data by encrypting, via the processor, the privileged data using an encryption algorithm and a unique encryption key, and wherein the second data includes the encrypted data.
18. The method of clause 17, further including: transmitting, by a transceiver, the first data and the unique encryption key to a client database authorized to access the first data.
19. The method of clauses 13 to 18, wherein the first data is stored in a record associated with the subject.
20. The method of clauses 13 to 19, further including: analyzing, by the medical device, the first data; identifying a correlation between the data indicating the state of the medical device and the data indicating the use of the medical device during the rescue event; and transmitting an indication of the correlation to the external device.
21. A server including: a processor configured to: receive, from a fleet of defibrillators, de-identified data associated with operation of the fleet of defibrillators when monitoring multiple subjects and treating the multiple subjects, the de-identified data omitting identifying information associated with the multiple subjects; extract, from the de-identified data, user operational data indicating states of the fleet of defibrillators; determine, using a computing model, a trend of misuse of the fleet of defibrillators by analyzing the user operational data; in response to determining the trend of misuse of the fleet of defibrillators, generating a software update that addresses the misuse, the software update including a change in a configuration setting of the defibrillators; and output, to the fleet of defibrillators, the software update.
22. The server of clause 21, wherein the user operational data indicates: alerts output to users of the defibrillators; signals detected by the defibrillators from the users, the signals including touch signals, button pushes, and voice commands; and accessory devices connected to the defibrillators.
23. The server of clauses 21 or 22, wherein the processor is further configured to: extract, from the de-identified data, patient data indicating physiological parameters detected by the defibrillators and electrical shocks output by the defibrillators; and extract, from the de-identified data, device data indicating ambient sounds detected by the defibrillators, motion detected by the defibrillators, moisture detected by the defibrillators, temperature detected by the defibrillators, electromagnetic interference detected by the defibrillators, locations of the defibrillators, and results of self-tests performed by the defibrillators, and the processor is configured to further determine, using the computing model, the trend of misuse of the fleet of defibrillators by analyzing the patient data and the device data.
24. The server of clauses 21 to 23, wherein determining, using the computing model, the trend of misuse includes: identifying multiple instances of greater than a threshold delay between a recommended treatment and a user-administered treatment; or identifying multiple instances of administration of non-indicated treatments.
25. A server including: a processor configured to: receive, from a fleet of medical devices, de-identified data associated with operation of the fleet of medical devices when monitoring or treating multiple subjects, the de-identified data omitting identifying information associated with the multiple subjects; extract, from the de-identified data, user operational data indicating states of the fleet of medical devices; determine, using a computing model, a trend indicating misuse of the fleet of medical devices by analyzing the user operational data; generate a software update that addresses the misuse; and output the software update to the fleet of medical devices.
26. The server of clause 25, wherein the fleet of medical devices include monitor-defibrillators, automated external defibrillators (AEDs), mechanical chest compression devices, or patient monitors.
27. The server of clause 25 or 26, wherein the user operational data indicates: output signals provided by the fleet of medical devices; and user input signals detected by the fleet of medical devices.
28. The server of clauses 25 to 27, wherein the trend indicating misuse of the fleet of medical devices includes: delays between treatment recommendations output by the fleet of medical devices and treatments administered to the multiple subjects; or non-indicated treatments administered to the multiple subjects.
29. The server of clauses 25 to 28, wherein: the processor is further configured to extract, from the de-identified data, patient data including physiological parameters of the multiple subjects or treatments administered to the multiple subjects, and the processor is configured to determine, using the computing model, the trend indicating misuse of the fleet of medical devices further by analyzing the patient data.
30. The server of clauses 25 to 29, wherein: the processor is further configured to extract, from the de-identified data, device data indicating environmental conditions of the fleet of medical devices or energy consumption of the fleet of medical devices, and the processor is configured to determine, using the computing model, the trend indicating misuse of the fleet of medical devices further by analyzing the device data.
31. The server of clauses 25 to 30, wherein: a portion of the fleet of medical devices include chest compression devices, and the user operational data indicates suction cup detachment events; and the software update includes instructions to: detect suction cup detachment events; and in response to detecting the suction cup detachment events, output alerts.
32. The server of clauses 25 to 31, wherein the computing model includes a machine learning model configured to receive the user operational data as input and output the trend indicating the misuse of the fleet of medical devices.
33. The server of clauses 25 to 32, wherein the software update includes instructions that, when executed by one of the medical devices, causes the fleet of medical device to: output a new alert in response to detecting an event; change a location of a visual alert on a screen of the fleet of medical device; change a volume or tone of an auditory alert output by a speaker of the fleet of medical device; or refrain from outputting an existing alert.
34. A method including: receiving, from a fleet of medical devices, de-identified data associated with operation of the fleet of medical devices when monitoring or treating multiple subjects, the de-identified data omitting identifying information associated with the multiple subjects; extracting, from the de-identified data, user operational data indicating states of the fleet of medical devices; determining, using a computing model, a trend indicating misuse of the fleet of medical devices by analyzing the user operational data; generating a software update that addresses the misuse; and outputting the software update to the fleet of medical devices.
35. The method of clause 34, wherein the trend is a first trend, the method further includes: extracting, from the de-identified data, device data indicating environmental conditions of the medical devices or energy consumption of the medical devices, and determining, using the computing model, a second trend indicating misuse of the fleet of medical devices further by analyzing the device data.
36. The method of clause 34 or 35, wherein the trend is a first trend, the method further includes extracting, from the de-identified data, patient data including physiological parameters of the subjects or treatments administered to the subjects, and determining, using the computing model, the trend indicating misuse of the fleet of medical devices further by analyzing the patient data.
37. The method of clauses 34 to 36, wherein the user operational data indicates: alerts output to users of the fleet of medical devices; signals detected by the fleet of medical devices from the users, the signals including touch signals, button pushes, and voice commands; and accessory devices connected to the fleet of medical devices.
38. The method of clauses 34 to 37, wherein outputting the software update to the fleet of medical devices includes: outputting a new alert in response to detecting an event; changing a location of a visual alert on a screen of the medical device; changing a volume or tone of an auditory alert output by a speaker of the medical device; or refraining from outputting an existing alert.
39. The method of clauses 34 to 38, wherein the trend indicating misuse of the medical devices includes: delays between treatment recommendations output by the medical devices and treatments administered to the subjects; or non-indicated treatments administered to the subjects.
40. The method of clauses 34 to 39, wherein a portion of the fleet of medical devices include chest compression devices, and the user operational data indicates suction cup detachment events; and the software update includes instructions to: detect suction cup detachment events; and in response to detecting the suction cup detachment events, output alerts to the portion of the fleet of medical devices including the chest compression devices.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain implementations are described herein, including the best mode known to the inventors for carrying out implementations of the disclosure. Of course, variations on these described implementations will become apparent to those of ordinary skill in the art upon reading the foregoing description. The inventors expect skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

What is claimed is:

1. A system, comprising:
a monitor-defibrillator comprising:
a monitoring circuit configured to detect an electrocardiogram (ECG) of a subject during a rescue event;
a parameter sensor configured to detect a physiological parameter of the subject;
a display configured to output a visual representation of the ECG and a visual representation of the physiological parameter;
an input device configured to receive an input signal from a user;
a treatment circuit configured to output an electrical shock to a heart of the subject in response to the input device receiving the input signal from the user;
memory;
a transceiver; and
a processor configured to:
generate first data comprising:
identifying data comprising an identifier of the subject, a location of the monitor-defibrillator, a time of the rescue event, or an identifier of the user;
parameter data indicating the ECG of the subject and the physiological parameter of the subject; and
operational data indicating the input signal and the electrical shock;
cause the memory to store the first data in a record associated with the subject;
generate second data by de-identifying the first data, the second data comprising the parameter data and the operational data and omitting the identifying data; and
cause the transceiver to transmit the second data; and
a computing device configured to:
receive the second data;
receive third data from multiple medical devices, the third data being de-identified;
identify a trend associated with the second data and the third data; and
in response to identifying the trend, output an alert.

2. The system of claim 1, wherein the monitor-defibrillator further comprises an environmental sensor configured to detect an environmental parameter indicative of an environment surrounding the monitor-defibrillator during the rescue event, the environmental sensor comprising:
a temperature sensor configured to measure a temperature of the environment external to the monitor-defibrillator; or
a humidity sensor configured to measure a humidity of the environment external to the monitor-defibrillator.

3. The system of claim 1, wherein de-identifying the first data comprises:
identifying, via the processor associated with the monitor-defibrillator, privileged data; and
generating encrypted data by encrypting, via the processor, the privileged data using an encryption algorithm and a unique encryption key, and
wherein the second data comprises the encrypted data.

4. A medical device, comprising:
a physiological sensor configured to detect a physiological parameter of a subject during a rescue event;
an environmental sensor configured to detect an environmental parameter indicating a state of an environment surrounding the medical device during the rescue event;
an input device configured to detect an input signal from a user;
memory; and
a processor configured to:
generate first data comprising:
an identifier of the subject or the rescue event; and
data indicating the physiological parameter of the subject, data indicating the environmental parameter of the medical device, or data indicating the input signal from the user;
cause the memory to store the first data;
generate second data by de-identifying the first data, the second data omitting the identifier of the subject or the rescue event; and
output the second data to an external device configured to:
receive de-identified data from a fleet of medical devices;
process the de-identified data; and
identify a trend associated with the de-identified data.

5. The medical device of claim 4, wherein the environmental sensor comprises:
a temperature sensor configured to measure a temperature of an environment external to the medical device; or
a humidity sensor configured to measure a humidity of the environment external to the medical device.

6. The medical device of claim 4, wherein the input signal from the user comprises data input by the user, a user response to an alert generated by the medical device, a time at which the user response was detected, a user response to a prompt generated by the medical device, or an indication of a selection of one or more user interface elements of the medical device.

7. The medical device of claim 4, wherein the identifier of the subject comprises a subject name, subject identification number, sex, age, weight, medical history, or physiological data.

8. The medical device of claim 4, wherein the first data further comprises a state of the medical device, the state comprising a model number, a serial number, a number of leads connected to the medical device, a type of lead connected to the medical device, a battery life, a volume level, a condition of one or more accessories associated with the medical device, data indicating the medical device is paired to a separate medical device, physiological parameter data, measurement reliability data, shock advisory data, audio output data, display output data, prompt data, alarm data, error data, failed acquisition data, or device motion during operation.

9. The medical device of claim 4, wherein de-identifying the first data comprises:
identifying, via the processor associated with the medical device, privileged data in the identifier; and
generating encrypted data by encrypting, via the processor, the privileged data using an encryption algorithm and a unique encryption key, and
wherein the second data comprises the encrypted data.

10. The medical device of claim 4, wherein the data is stored in a record associated with the subject.

11. The medical device of claim 4, further comprising:
a transceiver configured to transmit the first data to a client device operated by an authorized user.

12. The medical device of claim 4, wherein the medical device comprises a monitor-defibrillator, an automated external defibrillator (AED), a mechanical chest compression device, or a patient monitor.

13. A method performed by a medical device, the method comprising:
generating, at the medical device, first data comprising:
an identifier of a subject being monitored or treated by the medical device;
data indicating a state of the medical device during a rescue event; and
data indicating a use of the medical device during the rescue event;
storing the first data in a memory associated with the medical device;
generating second data by de-identifying, by a processor associated with the medical device, the first data; and
transmitting the second data to an external device configured to:
receive de-identified data from a fleet of medical devices; and
identify a trend associated with the de-identified data.

14. The method of claim 13, wherein the identifier comprises a subject name, subject identification number, sex, age, weight, medical history, or physiological data.

15. The method of claim 13, wherein the data indicating the state of the medical device comprises a model number, a serial number, a number of leads connected to the medical device, a type of lead connected to the medical device, a battery life, a volume level, a condition of one or more accessories associated with the medical device, physiological parameter data, shock advisory data, audio output data, display output data, or device motion during operation.

16. The method of claim 13, wherein the data indicating the use of the medical device comprises data input by a user, a user response to an alert generated by the medical device, a time at which the user response was detected, a user response to a prompt generated by the medical device, or an indication of a selection of one or more user interface elements of the medical device.

17. The method of claim 13, wherein de-identifying the first data comprises:
identifying, via the processor associated with the medical device, privileged data in the identifier; and
generating encrypted data by encrypting, via the processor, the privileged data using an encryption algorithm and a unique encryption key, and
wherein the second data comprises the encrypted data.

18. The method of claim 17, further comprising:
transmitting, by a transceiver, the first data and the unique encryption key to a client database authorized to access the first data.

19. The method of claim 13, wherein the first data is stored in a record associated with the subject.

20. The method of claim 13, further comprising:
analyzing, by the medical device, the first data;
identifying a correlation between the data indicating the state of the medical device and the data indicating the use of the medical device during the rescue event; and
transmitting an indication of the correlation to the external device.
